SEQUENCE LISTING (4 sequences)

SEQ ID NO: 1 - DNA, 2136 bp, Artificial Sequence
  Artificially synthesized CAase base sequence
  CDS (1)..(2136)
atggctgacctgctgccgaccgtgaaagtgtccgacctgccgaccgcaaccgaatcgtttgaaggcgactatctggtggtggatcaaagcgatgcgacgcgtaaaagtacctggtccgacatgttttcacgtttcggcctgatgcgcctgttttcgttccaggaaggcggtaccctggttagcccgaaagatcaagtcatcgaccgttctacgaaccgcatttatcagtggaccggtgcctacccgaaactggtgccggcagattctaccccggaaaccacgggcggtgttggcgagggtgcgtggtcagccaacgatccgagcctgcgtggtgacctggccggtgcaaatggctcaacgtttattggcggtccggccggtaccgtggcacagagcctggatggttttgttaccccggcccaattcatgggtaaatatccgaccacgaccgaagcagtcaccgctctggcggcctacgcaaaagaaaacaagaaagcggtgctggcctggggctggaatctggttctggaaaccagtgtctatattgatggtgtggaatggtacggcggttcctttaaccaaacgggcggtaatcgttatatgtacctgtcaaactcgacctttcgctgggttacgttcaccggcgtctgcacgcgtcattatggcggtcgcctgattatcaccgatagctcttgggttaacaatacgaataccgcagctatgctgctgcaggcgctgccgatcgaaggtaccatcgatattctggatagtgactttcgtggctgcaaatatggtattctgcagcagggtaccggtgcactggtgacccgtgcacgttttgctcgtctgaacttcaatgatctgaccggcgacgcgattgaatgtaatgtggttcagcgccattacaaagccggcggtctgacgatcgaagatatcaacatcgacaacatcaacaacaccgataactctccgaattggggcatcggtattggcgttgcgggtcaaggcccgtatggcgcaaacgctagcgatgaccagtacgtgtctggtattatcattcgtaatgtgaaaatgcgtcgcgttcgccagtgcatccactttgaactgtgtcgtgatttcaaagttgaaaacgttgaagtctatccggacgcaagcgtctctaatggtaccctgctggctagcggcggtgtcgtgtgctatggttgtaaagattacatcattgacggtgtccgtggcgaaatggtgaacggcgcgacccgctttatttatttcggttggggcgttaatcagggcacgtttgccgcaccgtgccgtgatttcaccctgcgtaacgtgcgcacgcataccggcctggtcgacatcccggtgtcagcgatggatgactggacgaatgatgtgaaagttgaagacattgaatgccacacctttaaatatcgtggtctggtgtcgaaactgcgtctggcagatatccgctgtaaacaattcgatggtattggcgactatgaagcgggtcagggtgaagcgggcggtgcaatgaaacgctgggcatggtgcagtgctgaaatcattaacatcaattccctggatgacaacggcgtcgccaatggtaaatttggccaagtgggtttcgatcatctgacgacctatggttgtaactttgacgttgtccagcacagtaaaaccaacggcaatcgtggtgttattctgctgaacgcaggcaacatctacatctccgataacgatgacttcccgcagggcaaagaattcgtgaaaggtgacatcattctgaagaaaaccggcggcatgtttgtggttgaaaccggcggtagctatatcgaaccgaacgatttcattaaagcaacggtcgtgggctctaaaaccatcgaatgtgcagctgatagttccattcgtcaaccgtgggcaacgcgcgcttttaaaagcgcgggtctgcagctgaccatcccgggtgccggtccgggcggtgcagatctgcagacgaccgtgatccgcgcaccgtatcaaaaaggtgcttggattacgccgttctacctggatatcgcggacccgattcagaccgcaaccccggataatacggcactggtctctacgaacccggtggtctactcagaacgcacg

SEQ ID NO: 2 - protein, 712 aa, Artificial Sequence
  Synthetic Construct
MADLLPTVKVSDLPTATESFEGDYLVVDQSDATRKSTWSDMFSRFGLMRLFSFQEGGTLVSPKDQVIDRSTNRIYQWTGAYPKLVPADSTPETTGGVGEGAWSANDPSLRGDLAGANGSTFIGGPAGTVAQSLDGFVTPAQFMGKYPTTTEAVTALAAYAKENKKAVLAWGWNLVLETSVYIDGVEWYGGSFNQTGGNRYMYLSNSTFRWVTFTGVCTRHYGGRLIITDSSWVNNTNTAAMLLQALPIEGTIDILDSDFRGCKYGILQQGTGALVTRARFARLNFNDLTGDAIECNVVQRHYKAGGLTIEDINIDNINNTDNSPNWGIGIGVAGQGPYGANASDDQYVSGIIIRNVKMRRVRQCIHFELCRDFKVENVEVYPDASVSNGTLLASGGVVCYGCKDYIIDGVRGEMVNGATRFIYFGWGVNQGTFAAPCRDFTLRNVRTHTGLVDIPVSAMDDWTNDVKVEDIECHTFKYRGLVSKLRLADIRCKQFDGIGDYEAGQGEAGGAMKRWAWCSAEIININSLDDNGVANGKFGQVGFDHLTTYGCNFDVVQHSKTNGNRGVILLNAGNIYISDNDDFPQGKEFVKGDIILKKTGGMFVVETGGSYIEPNDFIKATVVGSKTIECAADSSIRQPWATRAFKSAGLQLTIPGAGPGGADLQTTVIRAPYQKGAWITPFYLDIADPIQTATPDNTALVSTNPVVYSERT

SEQ ID NO: 3 - DNA, 2136 bp, Artificial Sequence
  Artificially synthesized CAase coding sequence
  CDS (1)..(2136)
  misc_feature (850)..(964): r is a or g
  misc_feature (919)..(921): nnn is ctg or gac
atggctgacctgctgccgaccgtgaaagtgtccgacctgccgaccgcaaccgaatcgtttgaaggcgactatctggtggtggatcaaagcgatgcgacgcgtaaaagtacctggtccgacatgttttcacgtttcggcctgatgcgcctgttttcgttccaggaaggcggtaccctggttagcccgaaagatcaagtcatcgaccgttctacgaaccgcatttatcagtggaccggtgcctacccgaaactggtgccggcagattctaccccggaaaccacgggcggtgttggcgagggtgcgtggtcagccaacgatccgagcctgcgtggtgacctggccggtgcaaatggctcaacgtttattggcggtccggccggtaccgtggcacagagcctggatggttttgttaccccggcccaattcatgggtaaatatccgaccacgaccgaagcagtcaccgctctggcggcctacgcaaaagaaaacaagaaagcggtgctggcctggggctggaatctggttctggaaaccagtgtctatattgatggtgtggaatggtacggcggttcctttaaccaaacgggcggtaatcgttatatgtacctgtcaaactcgacctttcgctgggttacgttcaccggcgtctgcacgcgtcattatggcggtcgcctgattatcaccgatagctcttgggttaacaatacgaataccgcagctatgctgctgcaggcgctgccgatcgaaggtaccatcgatattctggatagtgactttcgtggctgcaaatatggtattctgcagcagggtaccggtgcactggtgacccgtgcacgttttgctcgtctgracttcaatratctgaccggcracgcgattgaatgtaatgtggttcagcgccattacaaagccggcggtnnnacgatcgaaratatcracatcracaacatcaacaacaccgatractctccgaattggggcatcggtattggcgttgcgggtcaaggcccgtatggcgcaaacgctagcgatgaccagtacgtgtctggtattatcattcgtaatgtgaaaatgcgtcgcgttcgccagtgcatccactttgaactgtgtcgtgatttcaaagttgaaaacgttgaagtctatccggacgcaagcgtctctaatggtaccctgctggctagcggcggtgtcgtgtgctatggttgtaaagattacatcattgacggtgtccgtggcgaaatggtgaacggcgcgacccgctttatttatttcggttggggcgttaatcagggcacgtttgccgcaccgtgccgtgatttcaccctgcgtaacgtgcgcacgcataccggcctggtcgacatcccggtgtcagcgatggatgactggacgaatgatgtgaaagttgaagacattgaatgccacacctttaaatatcgtggtctggtgtcgaaactgcgtctggcagatatccgctgtaaacaattcgatggtattggcgactatgaagcgggtcagggtgaagcgggcggtgcaatgaaacgctgggcatggtgcagtgctgaaatcattaacatcaattccctggatgacaacggcgtcgccaatggtaaatttggccaagtgggtttcgatcatctgacgacctatggttgtaactttgacgttgtccagcacagtaaaaccaacggcaatcgtggtgttattctgctgaacgcaggcaacatctacatctccgataacgatgacttcccgcagggcaaagaattcgtgaaaggtgacatcattctgaagaaaaccggcggcatgtttgtggttgaaaccggcggtagctatatcgaaccgaacgatttcattaaagcaacggtcgtgggctctaaaaccatcgaatgtgcagctgatagttccattcgtcaaccgtgggcaacgcgcgcttttaaaagcgcgggtctgcagctgaccatcccgggtgccggtccgggcggtgcagatctgcagacgaccgtgatccgcgcaccgtatcaaaaaggtgcttggattacgccgttctacctggatatcgcggacccgattcagaccgcaaccccggataatacggcactggtctctacgaacccggtggtctactcagaacgcacg

SEQ ID NO: 4 - protein, 712 aa, Artificial Sequence
  misc_feature (284)..(284): The ′Xaa′ at location 284 stands for Asp, or Asn.
  misc_feature (287)..(287): The ′Xaa′ at location 287 stands for Asp, or Asn.
  misc_feature (291)..(291): The ′Xaa′ at location 291 stands for Asp, or Asn.
  misc_feature (307)..(307): The ′Xaa′ at location 307 stands for Leu, or Asp.
  misc_feature (311)..(311): The ′Xaa′ at location 311 stands for Asp, or Asn.
  misc_feature (313)..(313): The ′Xaa′ at location 313 stands for Asp, or Asn.
  misc_feature (315)..(315): The ′Xaa′ at location 315 stands for Asp, or Asn.
  misc_feature (322)..(322): The ′Xaa′ at location 322 stands for Asp, or Asn.
  Synthetic Construct
MADLLPTVKVSDLPTATESFEGDYLVVDQSDATRKSTWSDMFSRFGLMRLFSFQEGGTLVSPKDQVIDRSTNRIYQWTGAYPKLVPADSTPETTGGVGEGAWSANDPSLRGDLAGANGSTFIGGPAGTVAQSLDGFVTPAQFMGKYPTTTEAVTALAAYAKENKKAVLAWGWNLVLETSVYIDGVEWYGGSFNQTGGNRYMYLSNSTFRWVTFTGVCTRHYGGRLIITDSSWVNNTNTAAMLLQALPIEGTIDILDSDFRGCKYGILQQGTGALVTRARFARLXFNXLTGXAIECNVVQRHYKAGGXTIEXIXIXNINNTDNSPNWGIGIGVAGQGPYGANASDDQYVSGIIIRNVKMRRVRQCIHFELCRDFKVENVEVYPDASVSNGTLLASGGVVCYGCKDYIIDGVRGEMVNGATRFIYFGWGVNQGTFAAPCRDFTLRNVRTHTGLVDIPVSAMDDWTNDVKVEDIECHTFKYRGLVSKLRLADIRCKQFDGIGDYEAGQGEAGGAMKRWAWCSAEIININSLDDNGVANGKFGQVGFDHLTTYGCNFDVVQHSKTNGNRGVILLNAGNIYISDNDDFPQGKEFVKGDIILKKTGGMFVVETGGSYIEPNDFIKATVVGSKTIECAADSSIRQPWATRAFKSAGLQLTIPGAGPGGADLQTTVIRAPYQKGAWITPFYLDIADPIQTATPDNTALVSTNPVVYSERT

(19) United States
(12) Patent Application Publication
(10) Pub. No.: US 2021/0340187 A1
(43) Pub. Date: Nov. 4, 2021

(54) DESIGNED, ENZYMATIC BIOCIDE FOR REMOVAL OF FOODBORNE MICROBIAL CONTAMINATION

(71) Applicant: University of Virginia Patent Foundation, Charlottesville, VA (US)

(72) Inventors: Bryan Berger, Charlottesville, VA (US); Holly Mayton, Charlottesville, VA (US)

(73) Assignee: University of Virginia Patent Foundation, Charlottesville, VA (US)

(21) Appl. No.: 17/306,623

(22) Filed: May 3, 2021

Related U.S. Application Data
(60) Provisional application No. 63/018,951, filed on May 1, 2020.

(51) Int. Cl. C07K 14/01; A61L 2/16; A01N 37/46

(52) U.S. Cl. CPC C07K 14/01; A01N 37/46; A61L 2/16

(57) ABSTRACT

Provided are polypeptides that have at least about 95% but less than 100% sequence identity to SEQ ID NO: 2, optionally wherein the polypeptide has an amino acid sequence as set forth in SEQ ID NO: 4, with the proviso that the polypeptide does not have 100% sequence identity to SEQ ID NO: 2. Also provided are polypeptides that include an amino acid sequence that is a variant of SEQ ID NO: 2, wherein the variant sequence has at least one substitution at an amino acid position selected from the group consisting of D287, D291, D311, N313, D315, L307, and N284 of SEQ ID NO: 2; optionally wherein the polypeptide inhibits growth of a microbe and/or microbial biofilm and/or disrupts a microbial biofilm; nucleic acid molecules encoding the disclosed polypeptides; vectors and recombinant host cells that include the disclosed nucleic acid molecules; antimicrobial compositions that include an effective amount of the disclosed polypeptides, optionally that also include a carrier and/or one or more additional active agents; and methods for inhibiting the growth of microbes and/or microbial biofilms on surfaces and/or for disrupting microbial biofilms on surfaces and methods for inhibiting the growth of microbes on and/or in agricultural products and/or subjects.

### CROSS REFERENCE TO RELATED APPLICATION

This application claims priority to U.S. Provisional Application Ser. No. 63/018,951 filed May 1, 2020, the disclosure of which is incorporated by reference in its entirety herein.

### GOVERNMENT INTEREST

This invention was made with government support under federal grant number NSF 1801612 awarded by the National Science Foundation. The government has certain rights in the invention.

### TECHNICAL FIELD

The presently disclosed subject matter relates in some embodiments to a designed, enzymatic biocide for post-harvest removal of food pathogens and other antimicrobial method and compositions.

### BACKGROUND

Food safety is a growing global challenge in which pathogens are estimated to cause 600 million illnesses and 420,000 deaths annually (Havelaar et al., 2015). Recent high-profile foodborne illness outbreaks associated with leafy greens have raised public awareness about the serious health risks of improper food handling, processing, and packaging, as well as drive increasing demands to develop safe and effective solutions to prevent future outbreaks (Slayton et al., 2013; Herman et al., 2015; Sharapov et al., 2016). Biofilm formation on produce is one of the main causes of post-harvest pathogen persistence that leads to foodborne illnesses, as well as spoilage organism persistence that leads to product loss (Korber et al., 2009; Blaschek et al., 2015).

Biofilm is a secreted matrix, made up largely of polysaccharides, nucleic acids and proteins, which encapsulates bacteria cells and protects them from chemical and mechanical disruption, as well as enables adhesion to food, equipment, and packaging surfaces (Ryu et al., 2004; Ryu & Beuchat, 2005; Maharj an et al., 2017). Biofilms have been shown to protect cells from chlorine, the most commonly employed disinfectant in the produce safety industry (Corcoran et al., 2014; Meireles et al., 2017).

Increasingly, the fresh produce industry is pursing alternatives to bleach and other antimicrobials as bacteria have demonstrated to capacity to resist (Hoff & Akin, 1986). Currently used chemical sanitizers, including bleach, hydrogen peroxide, and peracetic acid, are also restricted in their use due to environmental and public health concerns, as well as customer preferences for organic, minimally-processed materials (Suslow, 2000; Olmez & Kretzschmar, 2009). Although a diversity of alternatives to bleach have been proposed and developed, there are still significant limitations in terms of their efficacy against biofilms. For example, ultraviolet (UV) irradiation is an effective method for eliminating bacteria on produce surfaces during packaging, but still does not kill bacteria embedded in protective biofilms (Elasri & Miller, 1999). Additionally, UV radiation and some organic chemical treatments can significantly affect food texture, taste, and appearance, presenting a challenge to consumer acceptance (Martinez-Sanchez et al., 2006; Duncan & Chang, 2012). High-pressure processing (HPP) is also an effective technique for sterilization that preserves food texture, flavor, and appearance, but is demonstratively less effective at removing biofilm-embedded pathogens.

Recently, enzymes have gained attention as alternatives to chemical disinfectants due to their ability to directly degrade components present in microbial biofilms, act specifically on biofilm without modifying food properties, and function under ambient conditions in water without a need for high temperatures, pressures, or chemical sanitizers. Dispersin B is one such example; it is a glycosyl hydrolase that degrades poly-N-acetylglucoseamine (PNAG), which is a key polysaccharide found in biofilms formed by the oral pathogen Aggregatibacter actinomycetemcomitans. Other examples include alginate lyase AlgL from Pseudomonas aeruginosa and human DNAse I, both of which have been shown to be effective at removing P. aeruginosa biofilms from cystic fibrosis patients.

However, an enzymatic disinfectant that can be employed to prevent and remove microbial biofilm and/or surface polysaccharides remains an ongoing need in the art.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides polypeptides comprising, consisting essentially of, or consisting of an amino acid sequence with at least about 95% sequence identity to an amino acid sequence as set forth in SEQ ID NO: 2; provided however that that the polypeptide does not have 100% sequence identity to SEQ ID NO: 2. In some embodiments, a polypeptide of the presently disclosed subject matter is an isolated polypeptide.

In some embodiments, the presently disclosed subject matter provides polypeptides, optionally isolated polypeptides, comprising, consisting essentially of, or consisting of an amino acid sequence that is a variant of the amino acid sequence of a wild-type Salmonella phage sequence set forth in SEQ ID NO: 2. In some embodiments, the variant sequence comprises, consists essentially of, or consists of at least one substitution at an amino acid position selected from the group consisting of N284, D287, D291, L307, D311, N313, D315, and N322 of SEQ ID NO: 2. In some embodiments, the variant sequence comprises, consists essentially of, or consists of an amino acid sequence as set forth in SEQ ID NO: 4 or an inhibitory fragment thereof. In some embodiments, said polypeptide inhibits the growth of a microbe or microbial biofilm, and/or disrupts a microbial biofilm.

In some embodiments, the presently disclosed subject matter provides nucleic acid molecules encoding a polypeptide as disclosed herein. In some embodiments, the nucleic acid molecule comprises an operably linked promoter. In some embodiments, said nucleic acid molecule is a DNA segment, and the DNA segment and promoter are operably linked in a recombinant vector.

In some embodiments, the presently disclosed subject matter provides recombinant host cells comprising a nucleic acid molecule and/or a vector as disclosed herein.

In some embodiments, the presently disclosed subject matter provides antimicrobial compositions. In some embodiments, the antimicrobial compositions comprise, consist essentially of, or consist of an effective amount of a polypeptide as described herein and an acceptable carrier. In some embodiments, the polypeptide is present at a concentration in the range of from about 0.1 microgram per milliliter to about 100 milligrams per milliliter. In some embodiments, the antimicrobial composition has a pH in the range of from about 4.0 to about 9.0. In some embodiments, the antimicrobial composition has antimicrobial activity against a bacterium selected from the group consisting of E. coli, Salmonella, Pseudomonas, Listeria, Stenotrophomonas, and/or other pathogenic bacteria.

In some embodiments, the antimicrobial composition further comprises one or more active agents, optionally wherein the active agent(s) is/are selected from the group comprising an additional antimicrobial agent (such as an antibiotic or antifungal agent), a disinfectant (e.g., bleach), a pesticide, a fertilizer, an insecticide, an attractant, a sterilizing agent, an acaricide, a nematocide, an herbicide, and a growth regulator.

In some embodiments, the presently disclosed subject matter provides methods for inhibiting the growth of microbe and/or microbial biofilms on surfaces, and/or disrupting microbial biofilms on surfaces. In some embodiments, the methods comprise, consist essentially of, or consist of contacting a surface with an effective amount of an antimicrobial composition as described herein. In some embodiments, the surface is a surface of an agricultural product, the surface of a medical device, or a surface in a subject.

In some embodiments, the presently disclosed subject matter provides methods for inhibiting the growth of a microbe on or in an agricultural product or a subject. In some embodiments, the methods comprise, consist essentially of, or consist of contacting and/or administering an antimicrobial composition as described herein to the agricultural product or to the subject. In some embodiments, the microbe is a pathogenic bacterium, such as but not limited to E. coli, Salmonella, Pseudomonas, Listeria, and/or Stenotrophomonas. In some embodiments, the antimicrobial composition is contacted and/or administered before, in conjunction with, and/or after the agricultural product or the subject is contacted with or administered one or more other antimicrobial active agents. In some embodiments, the one or more antimicrobial active agents are selected from the group comprising additional antimicrobial agent such as but not limited to antibiotics and/or antifungal agents, disinfectants such as but not limited to bleach, pesticides, fertilizers, insecticides, attractants, sterilizing agents, acaricides, nematocides, herbicides, and/or growth regulators.

Accordingly, it is an object of the presently disclosed subject matter to provide compositions and methods for treating and preventing biofilms. This and other objects are achieved in whole or in part by the presently disclosed subject matter.

An object of the presently disclosed subject matter having been stated above, other objects and advantages of the presently disclosed subject matter will become apparent to those of ordinary skill in the art after a study of the following description of the presently disclosed subject matter and non-limiting Figures, Examples, Sequence Listing, and Appendix. The Figures, Examples, Sequence Listing, and Appendix form part of the instant disclosure.

### BRIEF DESCRIPTION OF THE FIGURES

FIG. 1 is an exemplary SD S-PAGE gel used to verify expression and purification of a recombinantly generated CAase enzyme.

FIG. 2 is a bar graph showing prevention of biofilm formation on polycarbonate with the addition of 100 ppm enzyme by E. coli 25922, E. coli O157:H7, and Salmonella typhimurium as measured by OD₆₀₀. Biofilm inhibition percentages were calculated based on control wells with no CAase, using OD₆₀₀ values after crystal violet biofilm staining and dissolution. Gray bars depict negative controls and hatched bars depict 0.1 mg·mL CAase. Error bars represent ± the standard error of at least three independent replicates. **: p<0.01.

FIG. 3 is a bar graph showing removal of E. coli 25922, E. coli O157:H7, and Salmonella typhimurium biofilms on polycarbonate with the addition of 100 ppm enzyme typhimurium as measured by OD₆₀₀. Biofilm removal percentages were calculated based on control wells rinsed with 10 mM KCl salt solution, using OD600 values after crystal violet biofilm staining and dissolution. Gray bars depict negative controls and hatched bars depict 0.1 mg·mL CAase. Error bars represent ± the standard error of at least three independent replicates. *: p<0.05; **: p<0.01.

FIG. 4 is a bar graph showing detachment of E. coli O157:H7 from spinach leaf surfaces at 0 ppb, 250 ppb, and 1000 ppb CAase quantified by mass transfer rate coefficients (m/s; bars) and total number of cells removed (percentage; square points). Error bars represent ± the standard error of at least three independent replicates.

FIG. 5 is a series of images of growth of Salmonella typhimurium, E. coli O157:H7, and E. coli 25922 on LB agar plates (1:10 dilutions of treated spinach) used to calculate CFUs and reduction in pathogen persistence. The top three images are a control (10 ppm bleach) and the bottom three images are spinach treated with 0.1 g/L CAase plus 10 ppm bleach. CFU reductions were 92.8%, 92.6%, and 92.1% for Salmonella typhimurium, E. coli O157:H7, and E. coli 25922, respectively.

FIG. 6 is a bar graph of relative hydrophobicity of E. coli 25922, E. coli O157:H7, and Salmonella typhimurium with and without treatment with 100 ppm CAase.

FIG. 7 is a series of negative-stain electron micrographs of E. coli cells with (left panel) and without (right panel) treatment with 0.1 mg/mL CAase enzyme. Bars in lower left corners of Figures represent 2 μm.

FIG. 8 is a graph of representative detachment curves for E. coli O157:H7 cells over a 30 minute rinse with 10 mM KCl containing 0 (squares), 250 (triangles), or 1000 (circles) ppb CAase.

### BRIEF DESCRIPTION OF THE SEQUENCE LISTING

SEQ ID NO: 1 is a nucleic acid sequence that encodes a wild-type CAase polypeptide of the presently disclosed subject matter.

SEQ ID NO: 2 is an amino acid sequence encoded by SEQ ID NO: 1.

SEQ ID NO: 3 is a nucleotide sequence showing non-limiting nucleotide substitutions that can be employed in the generation of CAases of the presently disclosed subject matter.

SEQ ID NO: 4 is an exemplary amino acid sequence encoded by SEQ ID NO: 3. In SEQ ID NO: 4, one or more of amino acid positions 284, 287, 291, 307, 311, 313, 315, and 322 can be modified. Although in SEQ ID NO: 4 these positions are listed as aspartic acid, leucine, or asparagine, other amino acid substitutions could also be introduced including but not limited to substitutions of glutamic acid and/or glutamine at any of these positions.

### DETAILED DESCRIPTION

Headings are included herein for reference and to aid in locating certain sections. These headings are not intended to limit the scope of the concepts described therein under, and these concepts may have applicability in other sections throughout the entire specification.

The presently disclosed subject matter will now be described more fully hereinafter with reference to the accompanying Figures, Examples, and Sequence Listing in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art. Certain components in the Figures, Examples, and Sequence Listing are not necessarily to scale, emphasis instead being placed upon illustrating the principles of the presently disclosed subject matter (in some cases schematically).

The presently disclosed subject matter relates at least in part to an enzymatic disinfectant that can be employed to prevent and remove microbial biofilm and surface polysaccharides. In some embodiments, a candidate enzyme, referred to as “CAase”, which has biofilm-degrading activity and stability to improve performance, was developed using a homology-based search based on glycosyl hydrolases with activity against bacterial biofilm. The results presented herein indicate the enzymatic effectiveness at disrupting mature biofilm formation and production, as well as initial bacterial attachment in a microfluidic model of rinsing produce surfaces.

### I. Definitions

While the following terms are believed to be well understood by one of ordinary skill in the art, the following definitions are set forth to facilitate explanation of the presently claimed subject matter.

In describing the presently disclosed subject matter, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the presently disclosed subject matter and the claims.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about”, when referring to a value or to an amount of a composition, mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

As used herein, the terms “administration of” and or “administering” a compound should be understood to mean providing a composition in accordance with the presently disclosed subject matter to a surface, a product, a subject, or other item or article in need of treatment. Administration of a composition of the presently disclosed subject matter to a subject by any number of routes is provided, including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, or rectal administration.

As used herein, the term “aerosol” refers to suspension in the air. In particular, aerosol refers to the particlization or atomization of a formulation of the presently disclosed subject matter and its suspension in the air.

The term “alterations in peptide structure” as used herein refers to changes including, but not limited to, changes in sequence, and post-translational modification.

As used herein, amino acids are represented by the full name thereof, by the three letter code corresponding thereto, or by the one-letter code corresponding thereto, as indicated in Table 1:

TABLE 1
Table of Amino Acids and
Functionally Equivalent Codons

| | 3- | 1- | |
|---|---|---|---|
| | Letter | Letter | |
| Amino Acid | Code | Code | Codons |
| Alanine | Ala | A | GCA; GCC; GCG; GCU |
| Cysteine | Cys | C | UGC; UGU |
| Aspartic Acid | Asp | D | GAC; GAU |
| Glutamic acid | Glu | E | GAA; GAG |
| Phenylalanine | Phe | F | UUC; UUU |
| Glycine | Gly | G | GGA; GGC; GGG; GGU |
| Histidine | His | H | CAC; CAU |
| Isoleucine | Ile | I | AUA; AUC; AUU |
| Lysine | Lys | K | AAA; AAG |
| Leucine | Leu | L | UUA; UUG; CUA; CUC; CUG; CUU |
| Methionine | Met | M | AUG |
| Asparagine | Asn | N | AAC; AAU |
| Proline | Pro | P | CCA; CCC; CCG; CCU |
| Glutamine | Gln | Q | CAA; CAG |
| Arginine | Arg | R | AGA; AGG; CGA; CGC; CGG; CGU |
| Serine | Ser | S | ACG; AGU; UCA; UCC; UCG; UCU |
| Threonine | Thr | T | ACA; ACC; ACG; ACU |
| Valine | Val | V | GUA; GUC; GUG; GUU |
| Tryptophan | Trp | W | UGG |
| Tyrosine | Tyr | Y | UAC; UAU |

The term “amino acid” is used interchangeably with “amino acid residue”, and may refer to a free amino acid and to an amino acid residue of a peptide. It will be apparent from the context in which the term is used whether it refers to a free amino acid or a residue of a peptide. Amino acids can be classified into seven groups on the basis of the side chain: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

The term “biocompatible”, as used herein, refers to a material that does not elicit a substantial detrimental response in the host.

As used herein, the term “biologically active fragments” or “bioactive fragment” of the polypeptides encompasses natural or synthetic portions of the full-length protein that are capable of specific binding to their natural ligand or of performing the function of the protein.

The term “biological sample”, as used herein, refers to samples obtained from a subject, including, but not limited to, sputum, mucus, phlegm, tissues, biopsies, cerebrospinal fluid, blood, serum, plasma, other blood components, gastric aspirates, throat swabs, pleural effusion, peritoneal fluid, follicular fluid, ascites, skin, hair, tissue, blood, plasma, cells, saliva, sweat, tears, semen, stools, Pap smears, and urine. One of skill in the art will understand the type of sample needed.

As used herein, the term “CAase” refers to a colanic acid degrading polypeptide of the presently disclosed subject matter. CAases of the presently disclosed subject matter are polypeptides comprising, consisting essentially of, or consisting of an amino acid sequence with at least about 95% sequence identity to an amino acid sequence as set forth in SEQ ID NO: 2, with the proviso that the polypeptide does not have 100% sequence identity to SEQ ID NO: 2. In some embodiments, the polypeptide has colonic acid degrading activity. Also encompassed within the definition of CAase are fragments of the presently disclosed subject matter polypeptides that themselves have colonic acid degrading activity. It is noted that a polypeptide that has an amino acid sequence as set forth in SEQ ID NO: 2 is itself is a colonic acid degrading enzyme, which is in some embodiments referred to herein as a “wild type CAase”.

As used herein, the term “carrier molecule” refers to any molecule that is chemically conjugated to a molecule of interest.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

The term “competitive sequence” refers to a peptide or a modification, fragment, derivative, or homolog thereof that competes with another peptide for its cognate binding site.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

As used herein, the term “conservative amino acid substitution” is defined herein as an amino acid exchange within one of the following five groups:

I. Small aliphatic, nonpolar or slightly polar residues: Ala, Ser, Thr, Pro, Gly;

II. Polar, negatively charged residues and their amides: Asp, Asn, Glu, Gln;

III. Polar, positively charged residues: His, Arg, Lys;

IV. Large, aliphatic, nonpolar residues: Met Leu, Ile, Val, Cys

V. Large, aromatic residues: Phe, Tyr, Trp

A “control” cell is a cell having the same cell type as a test cell. The control cell may, for example, be examined at precisely or nearly the same time the test cell is examined. The control cell may also, for example, be examined at a time distant from the time at which the test cell is examined, and the results of the examination of the control cell may be recorded so that the recorded results may be compared with results obtained by examination of a test cell.

A “test” cell is a cell being examined.

As used herein, a “derivative” of a compound refers to a chemical compound that may be produced from another compound of similar structure in one or more steps, as in replacement of H by an alkyl, acyl, or amino group.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health. A “condition” encompasses both diseases and disorders.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains.

As used herein, an “effective amount” or means an amount sufficient to produce a selected effect, such as inhibiting the growth of a microbe or a microbial biofilm, and/or disrupting a microbial biofilm, including on a surface. In the context of administering compositions in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “enhancer” is a DNA regulatory element that can increase the efficiency of transcription, regardless of the distance or orientation of the enhancer relative to the start site of transcription.

As used herein, an “essentially pure” preparation of a particular protein or peptide is a preparation wherein at least about 95%, and preferably at least about 99%, by weight, of the protein or peptide in the preparation is the particular protein or peptide.

A “subsequence”, “fragment” or “segment” is a portion of an amino acid sequence, comprising at least one amino acid, or a portion of a nucleic acid sequence comprising at least one nucleotide. The terms “subsequence”, “fragment” and “segment” are used interchangeably herein.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may be in some embodiments at least about 20 nucleotides in length, in some embodiments at least about 50 nucleotides, in some embodiments from about 50 to about 100 nucleotides, in some embodiments at least about 100 to about 200 nucleotides, in some embodiments at least about 200 to about 300 nucleotides, in some embodiments at least about 300 to about 350 nucleotides, in some embodiments at least about 350 to about 500 nucleotides, in some embodiments at least about 500 to about 600 nucleotides, in some embodiments at least about 600 to about 620 nucleotides, in some embodiments at least about 620 to about 650 nucleotides, and in some embodiments, the nucleic acid fragment is greater than about 650 nucleotides in length.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ ATTGCC5′ and 3′ TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin & Altschul, 1990, modified as in Karlin & Altschul, 1993. This algorithm is incorporated into the NBLAST and XBLAST programs of Altschul et al., 1990a, and can be accessed, for example at the National Center for Biotechnology Information (NCBI) world wide web site having the universal resource locator using the BLAST tool at the NCBI website. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997. Alternatively, PSI-Blast or PHI-Blast can be used to perform an iterated search which detects distant relationships between molecules (Altschul et al., 1997) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “inhibit”, as used herein, refers to the ability of a composition, agent, or method to reduce, prevent or impede a described function, level, activity, rate, etc., based on the context in which the term “inhibit” is used. In some embodiments, inhibition is by at least 10%, in some embodiments by at least 25%, in some embodiments by at least 50%, and in some embodiments, the function is inhibited by at least 75%. The term “inhibit” is used interchangeably with “reduce”, “prevent” and “block.” However, the term does not imply that each and every one of these functions must be inhibited at the same time.

An “isolated polypeptide” refers to a polypeptide, or segment or fragment thereof, which has been separated from a naturally occurring state and/or that is present in a substantially purified form. In some embodiments, an isolated polypeptide refers to a polypeptide that has been isolated from one or more substances otherwise present in an artificial reaction by which the polypeptide is produced or employed (e.g., an in vitro expression reaction).

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell and/or which might be otherwise present in an artificial reaction by which the nucleic acids are produced or employed. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions, e.g., a nucleic acid molecule that hybridizes to one complementary sequence at the 5′ end and to another complementary sequence at the 3′ end, thus joining two non-complementary sequences.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double-stranded DNA and cDNA. Furthermore, the terms, “nucleic acid”, “DNA”, “RNA” and similar terms also include nucleic acid analogs, i.e. analogs having other than a phosphodiester backbone. For example, the so-called “peptide nucleic acids”, which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term “nucleic acid” also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

The term “peptide” typically refers to short polypeptides or to peptides shorter than the full length native or mature protein.

As used herein, the term “carrier” means a chemical composition with which an appropriate compound or derivative can be combined and which, following the combination, can be used to administer the appropriate compound. In some embodiments, the carrier is pharmaceutically acceptable, including for pharmaceutically acceptable for use in humans. As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Pharmaceutically acceptable” means physiologically tolerable, for either human or veterinary application.

As used herein, “pharmaceutical compositions” include formulations for human and veterinary use. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the presently disclosed subject matter is provided include, but are not limited to, humans and other primates, mammals including commercially relevant mammals such as cattle, pigs, horses, sheep, cats, and dogs, birds including commercially relevant birds such as chickens, ducks, geese, and turkeys.

A pharmaceutical composition of the presently disclosed subject matter may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the presently disclosed subject matter may further comprise one or more additional pharmaceutically active agents. Controlled- or sustained-release formulations of a pharmaceutical composition of the presently disclosed subject matter may be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Genaro, 1985, which is incorporated herein by reference.

Typically, dosages of a composition of the presently disclosed subject matter which may be administered to an animal, in some embodiments a human, comprise an effective amount as described elsewhere herein. The precise dosage administered will vary depending upon any number of factors, including but not limited to, the type of animal and type of state being treated, the age of the animal and the route of administration.

The composition may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the condition or state being treated, the type and age of the animal, etc.

Suitable preparations include oral preparations, either as liquid solutions or suspensions, however, solid forms suitable for solution in, suspension in, liquid prior to administration, may also be prepared. The preparation may also be emulsified, or the polypeptides encapsulated in liposomes. The active ingredients are often mixed with excipients which are pharmaceutically acceptable and compatible with the active ingredient. Suitable excipients are, for example, water saline, dextrose, glycerol, ethanol, or the like and combinations thereof.

The presently disclosed subject matter also includes a kit comprising a composition of the presently disclosed subject matter and an instructional material which describes approaches for administering the composition. In another embodiment, this kit comprises a (optionally sterile) solvent suitable for dissolving or suspending the composition of the presently disclosed subject matter prior to administering the compound.

As used herein, an “instructional material” includes a publication, a recording, a diagram, or any other medium of expression which can be used to communicate the usefulness of the peptide of the presently disclosed subject matter in the kit for effecting alleviation of the various states recited herein. The instructional material of the kit of the presently disclosed subject matter may, for example, be affixed to a container which contains a composition of the presently disclosed subject matter or be shipped together with a container which contains the composition.

Alternatively, the instructional material may be shipped separately from the container with the intention that the instructional material and the composition be used cooperatively by the recipient.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. For example, a “preventive” or “prophylactic” treatment is a treatment administered to surface at risk for exposure to microbes, including exposure such that a biofilm might develop. By way of additional example, a “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. A “significant detectable level” is an amount of contaminate that would be visible in the presented data and would need to be addressed/explained during analysis of the forensic evidence.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

A “recombinant cell” is a cell that comprises a transgene. Such a cell may be a eukaryotic or a prokaryotic cell. Also, the transgenic cell encompasses, but is not limited to, an embryonic stem cell comprising the transgene, a cell obtained from a chimeric mammal derived from a transgenic embryonic stem cell where the cell comprises the transgene, a cell obtained from a transgenic mammal, or fetal or placental tissue thereof, and a prokaryotic cell comprising the transgene.

The term “regulate” refers to either stimulating or inhibiting a function or activity of interest.

As used herein, the term “reporter gene” means a gene, the expression of which can be detected using a known method. By way of example, the Escherichia coli lacZ gene may be used as a reporter gene in a medium because expression of the lacZ gene can be detected using known methods by adding the chromogenic substrate o-nitrophenyl-β-galactoside to the medium (Gerhardt et al., 1994).

A “sample”, as used herein, refers preferably to a biological sample from a subject for which an assay or other use is needed, including, but not limited to, normal tissue samples, diseased tissue samples, sputum, mucus, phlegm, biopsies, cerebrospinal fluid, blood, serum, plasma, other blood components, gastric aspirates, throat swabs, pleural effusion, peritoneal fluid, follicular fluid, ascites, skin, hair, tissue, blood, plasma, cells, saliva, sweat, tears, semen, stools, Pap smears, and urine. A sample can also be any other source of material obtained from a subject which contains cells, tissues, or fluid of interest. A sample can also be obtained from cell or tissue culture.

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

As used herein, the term “solid support” relates to a solvent insoluble substrate that is capable of forming linkages (preferably covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

By the term “specifically binds to”, as used herein, is meant when a compound or ligand functions in a binding reaction or assay conditions which is determinative of the presence of the compound in a sample of heterogeneous compounds.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, preferably a human.

As used herein, a “subject in need thereof” is a patient, animal, mammal, or human, who will benefit from a composition or a method of the presently disclosed subject matter.

As used herein, a “substantially homologous amino acid sequences” includes those amino acid sequences which have in some embodiments at least about 95% homology, in some embodiments at least about 96% homology, in some embodiments at least about 97% homology, in some embodiments at least about 98% homology, and in some embodiments at least about 99% or more homology to an amino acid sequence of a reference antibody chain. Amino acid sequence similarity or identity can be computed by using the BLASTP and TBLASTN programs which employ the BLAST (Basic Local Alignment Search Tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99%, or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: in some embodiments 7% sodium dodecyl sulfate SDS, 0.5 M NaPO₄, 1 mM EDTA at 50° C. with washing in 2× standard saline citrate (SSC), 0.1% SDS at 50° C.; in some embodiments in 7% (SDS), 0.5 M NaPO₄, 1 mM EDTA at 50° C. with washing in 1×SSC, 0.1% SDS at 50° C.; in some embodiments 7% SDS, 0.5 M NaPO₄, 1 mM EDTA at 50° C. with washing in 0.5×SSC, 0.1% SDS at 50° C.; and in some embodiments in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984), and the BLASTN or FASTA programs (Altschul et al., 1990a; Altschul et al., 1990b; Altschul et al., 1997). The default settings provided with these programs are suitable for determining substantial similarity of nucleic acid sequences for purposes of the presently disclosed subject matter.

The term “substantially pure” describes a compound, e.g., a protein or polypeptide which has been separated from components which naturally accompany it.

Typically, a compound is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, a “transgenic cell” is any cell that comprises a nucleic acid sequence that has been introduced into the cell in a manner that allows expression of a gene encoded by the introduced nucleic acid sequence.

The term to “treat”, as used herein, means exposing a surface, product, subject, and the like to an agent, such as an antimicrobial composition of the presently disclosed subject matter to effect a change in a state or condition of the surface, product, subject, and the like. In the case of a subject, it can mean reducing the frequency with which symptoms are experienced by a patient or subject or administering an agent or compound to reduce the frequency with which symptoms are experienced.

A “variant”, as described herein, refers to a peptide or polypeptide that differs from a reference peptide or polypeptide or to a segment of DNA that differs from the reference DNA. A “marker” or a “polymorphic marker”, as defined herein, is a variant. Alleles that differ from the reference are referred to as “variant” alleles.

A “vector” is a composition of matter which comprises a nucleic acid and which can be used to deliver the nucleic acid to the interior of a cell.

Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

As used herein, the term “substantially”, when referring to a value, an activity, or to an amount of a composition, mass, weight, temperature, time, volume, concentration, percentage, etc., is meant to encompass variations of in some embodiments ±40%, in some embodiments ±30%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

### II. Polypeptides of the Presently Disclosed Subject Matter

II.A. Generally

In some embodiments, provided is an isolated polypeptide comprising, consisting essentially of, or consisting of an amino acid sequence having at least about 95% sequence identity to a polypeptide having an amino acid sequence as set forth in SEQ ID NO: 2, provided however that that the polypeptide does not have 100% sequence identity to SEQ ID NO: 2. Exemplary such amino acid sequences are presented in SEQ ID NO: 4. In SEQ ID NO: 4, one or more of amino acid positions 284, 287, 291, 307, 311, 313, 315, and 322 are shown to be modified. Although in SEQ ID NO: 4 these positions are listed as aspartic acid, leucine, or asparagine, other amino acid substitutions can also be introduced at one or more of these positions. By way of example and not limitation, the amino acid at one or more of these positions of SEQ ID NO: 2 or SEQ ID NO: 4 can in some embodiments be substituted to a glutamic acid and/or glutamine.

In some embodiments, provided is an isolated polypeptide comprising an amino acid sequence that is a variant of the amino acid sequence of a wild-type Salmonella phage sequence set forth in SEQ ID NO: 2, wherein the variant sequence comprises at least one substitution at an amino acid position selected from the group consisting of N284, D287, D291, L307, D311, N313, D315, and N322 of SEQ ID NO: 2; and wherein said polypeptide inhibits the growth of a microbe or microbial biofilm, or degrades a microbial biofilm.

In some embodiments, a nucleic acid molecule encoding a polypeptide of the presently disclosed subject matter is provided. In some embodiments the nucleic acid molecule is positioned under the control of a promoter. Representative promoters are described elsewhere herein. In some embodiments, the nucleic acid molecule is a DNA segment, and the DNA segment and promoter are operationally linked in a recombinant vector. In some embodiments, a recombinant host cell, comprising the nucleic acid molecule or comprising the vector, is provided.

In some embodiments, an antimicrobial composition, comprising an effective amount of a polypeptide of the presently disclosed subject matter and an acceptable carrier is provided. In some embodiments, the antimicrobial composition further comprises one or more active agents. In some embodiments, the active agent(s) is/are selected from the group comprising an additional antimicrobial agent (such as an antibiotic or antifungal agent), a disinfectant (e.g., bleach), a pesticide, a fertilizer, an insecticide, an attractant, a sterilizing agent, an acaricide, a nematocide, an herbicide, and a growth regulator. In some embodiments, the antimicrobial composition is administered before, in conjunction, and after one or more active agents. In some embodiments, the active agent(s) is/are selected from the group comprising an additional antimicrobial agent (such as an antibiotic or antifungal agent), a disinfectant (e.g., bleach), a pesticide, a fertilizer, an insecticide, an attractant, a sterilizing agent, an acaricide, a nematocide, an herbicide, and a growth regulator.

In some embodiments, the polypeptide is present at a concentration in the range of from about 0.01 microgram per milliliter to about 10 milligrams per milliliter. In some embodiments, the antimicrobial composition has a pH in the range of from about 4.0 to about 9.0. In some embodiments, the antimicrobial composition has antimicrobial activity against E. coli, Salmonella, Pseudomonas, Listeria, Stenotrophomonas and/or other pathogenic bacteria.

II.B. Polypeptide Modification and Preparation

Recombinant DNA methodologies can be used to prepare proteins, polypeptides, and/or peptides of the presently disclosed subject matter. Representative such techniques are disclosed in the Examples set forth herein below. Additional techniques are disclosed in U.S. Pat. No. 7,989,604; herein incorporated by reference in its entirety.

The proteins, polypeptides or peptides of the presently disclosed subject matter may be readily prepared by standard, well-established techniques, such as solid-phase peptide synthesis (SPPS) as described by Stewart et al., 1984 and as described by Bodanszky & Bodanszky, 1984. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions that will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters.

Examples of solid phase peptide synthesis methods include the BOC method that utilized tert-butyloxcarbonyl as the α-amino protecting group, and the FMOC method which utilizes 9-fluorenylmethyloxcarbonyl to protect the α-amino of the amino acid residues, both methods of which are well-known by those of skill in the art.

To ensure that the proteins, polypeptides or peptides of the presently disclosed subject matter obtained from either chemical or biological synthetic techniques is the desired peptide, analysis of the peptide composition should be conducted. Such amino acid composition analysis may be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

Prior to its use, the proteins, polypeptides or peptides of the presently disclosed subject matter can be purified to remove contaminants. In this regard, it will be appreciated that the proteins, polypeptides or peptides of the presently disclosed subject matter will be purified to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C4-, C8- or C18-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

Substantially pure proteins, polypeptides or peptides of the presently disclosed subject matter obtained as described herein may be purified by following known procedures for protein purification, wherein an immunological, enzymatic or other assay is used to monitor purification at each stage in the procedure.

Protein purification methods are well known in the art, and are described, for example in Deutscher et al., 1990.

It will be appreciated, of course, that the proteins, polypeptides or peptides of the presently disclosed subject matter may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide.

For example, suitable N-terminal blocking groups can be introduced by alkylation or acylation of the N-terminus. Examples of suitable N-terminal blocking groups include C1-C5 branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH2), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Acid addition salts of the presently disclosed subject matter are also contemplated as functional equivalents. Thus, a protein, polypeptide or peptide of the presently disclosed subject matter in accordance with the presently disclosed subject matter treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of a protein, polypeptide or peptide of the presently disclosed subject matter is suitable for use.

Modifications (which do not normally alter primary sequence) include in vivo, or in vitro chemical derivatization of polypeptides, e.g., acetylation, or carboxylation.

Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are proteins, polypeptides or peptides which have been modified using ordinary molecular biological techniques so as to improve their resistance to proteolytic degradation or to optimize solubility properties or to render them more suitable as a therapeutic agent. Analogs of such proteins, polypeptides or peptides include those containing residues other than naturally occurring L-amino acids, e.g., D-amino acids or non-naturally occurring or non-standard synthetic amino acids. The proteins, polypeptides or peptides of the presently disclosed subject matter are not limited to products of any of the specific exemplary processes listed herein. The presently disclosed subject matter includes the use of beta-alanine (also referred to as β-alanine, β-Ala, bA, and PA.

II.C. Amino Acid Substitutions

In certain embodiments, the disclosed methods and compositions may involve preparing protein, polypeptides, and/or peptides with one or more substituted amino acid residues.

In various embodiments, the structural, physical and/or active characteristics of sequences may be optimized by replacing one or more amino acid residues.

Exemplary amino acid sequences with one or more substitutions relative to SEQ ID NO: 2 are presented in SEQ ID NO: 4. In SEQ ID NO: 4, one or more of amino acid positions 284, 287, 291, 307, 311, 313, 315, and 322 are shown to be modified. Although in SEQ ID NO: 4 these positions are listed as aspartic acid, leucine, or asparagine, other amino acid substitutions can also be introduced at one or more of these positions. By way of example and not limitation, the amino acid at one or more of these positions of SEQ ID NO: 2 or SEQ ID NO: 4 can in some embodiments be substituted to a glutamic acid and/or glutamine.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the presently disclosed subject matter are also contemplated, for example, inverted peptides in which all amino acids are substituted with D-amino acid forms.

The skilled artisan will be aware that, in general, amino acid substitutions in a peptide typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art.

For example, one can make the following isosteric and/or conservative amino acid changes in the parent polypeptide sequence with the expectation that the resulting polypeptides would have a similar or improved profile of the properties described above:

Substitution of alkyl-substituted hydrophobic amino acids: including alanine, leucine, isoleucine, valine, norleucine, S-2-aminobutyric acid, S-cyclohexylalanine or other simple alpha-amino acids substituted by an aliphatic side chain from C1-10 carbons including branched, cyclic and straight chain alkyl, alkenyl or alkynyl substitutions.

Substitution of aromatic-substituted hydrophobic amino acids: including phenylalanine, tryptophan, tyrosine, biphenylalanine, 1-naphthylalanine, 2-naphthylalanine, 2-benzothienylalanine, 3-benzothienylalanine, histidine, amino, alkylamino, dialkylamino, aza, halogenated (fluoro, chloro, bromo, or iodo) or alkoxy-substituted forms of the previous listed aromatic amino acids, illustrative examples of which are: 2-,3- or 4-aminophenylalanine, 2-,3- or 4-chlorophenylalanine, 2-,3- or 4-methylphenylalanine, 2-,3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2,3, or 4-biphenylalanine, 2′,-3′,- or 4′-methyl-2, 3 or 4-biphenylalanine, and 2- or 3-pyridylalanine.

Substitution of amino acids containing basic functions: including arginine, lysine, histidine, ornithine, 2,3-diaminopropionic acid, homoarginine, alkyl, alkenyl, or aryl-substituted (from C1-C10 branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma′-diethyl-homoarginine. Included also are compounds such as alpha methyl arginine, alpha methyl 2,3-diaminopropionic acid, alpha methyl histidine, alpha methyl ornithine where alkyl group occupies the pro-R position of the alpha carbon. Also included are the amides formed from alkyl, aromatic, heteroaromatic (where the heteroaromatic group has one or more nitrogens, oxygens, or sulfur atoms singly or in combination) carboxylic acids or any of the many well-known activated derivatives such as acid chlorides, active esters, active azolides and related derivatives) and lysine, ornithine, or 2,3-diaminopropionic acid.

Substitution of acidic amino acids: including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids.

Substitution of side chain amide residues: including asparagine, glutamine, and alkyl or aromatic substituted derivatives of asparagine or glutamine.

Substitution of hydroxyl containing amino acids: including serine, threonine, homoserine, 2,3-diaminopropionic acid, and alkyl or aromatic substituted derivatives of serine or threonine. It is also understood that the amino acids within each of the categories listed above can be substituted for another of the same group.

For example, the hydropathic index of amino acids may be considered (Kyte & Doolittle, 1982). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within +/−2 is preferred, within +/−1 are more preferred, and within +/−0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, it would generally not be preferred to replace an amino acid with a compact side chain, such as glycine or serine, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see, e.g., Chou & Fasman, 1974; Chou & Fasman, 1978; Chou & Fasman, 1979).

Based on such considerations and extensive empirical study, tables of conservative amino acid substitutions have been constructed and are known in the art. For example: arginine and lysine; glutamate and aspartate; serine and threonine; glutamine and asparagine; and valine, leucine and isoleucine. Alternatively: Ala (A) Leu, Ile, Val; Arg (R) Gln, Asn, Lys; Asn (N) His, Asp, Lys, Arg, Gln; Asp (D) Asn, Glu; Cys (C) Ala, Ser; Gln (Q) Glu, Asn; Glu (E) Gln, Asp; Gly (G) Ala; His (H) Asn, Gln, Lys, Arg; Ile (I) Val, Met, Ala, Phe, Leu; Leu (L) Val, Met, Ala, Phe, Ile; Lys (K) Gln, Asn, Arg; Met (M) Phe, Ile, Leu; Phe (F) Leu, Val, Ile, Ala, Tyr; Pro (P) Ala; Ser (S), Thr; Thr (T) Ser; Trp (W) Phe, Tyr; Tyr (Y) Trp, Phe, Thr, Ser; Val (V) Ile, Leu, Met, Phe, Ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp (see e.g., PROWL Rockefeller University website). For solvent exposed residues, conservative substitutions would include: Asp and Asn; Asp and Glu; Glu and Gln; Glu and Ala; Gly and Asn; Ala and Pro; Ala and Gly; Ala and Ser; Ala, and Lys; Ser and Thr; Lys and Arg; Val and Leu; Leu and Ile; Ile and Val; Phe and Tyr. Various matrices have been constructed to assist in selection of amino acid substitutions, such as the PAM250 scoring matrix, Dayhoff matrix, Grantham matrix, McLachlan matrix, Doolittle matrix, Henikoff matrix, Miyata matrix, Fitch matrix, Jones matrix, Rao matrix, Levin matrix and Risler matrix.

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

As set forth herein, in some embodiments the polypeptides of the presently disclosed subject matter comprise, consist essentially of, or consist of an amino acid sequence that is at least 95% identical but less than 100% identical to SEQ ID NO: 2, or a subsequence thereof that has antimicrobial activity. Exemplary, non-limiting nucleotide/amino acid substitutions that can be present in the polypeptides of the presently disclosed subject matter as compared to SEQ ID NOs: 1 and 2 include those set forth in SEQ ID NOs: 3 and 4, which can be summarized as follows: A850G in SEQ ID NO: 1 (N284D in SEQ ID NOs: 2 and 4); G859A in SEQ ID NO: 1 (D287N in SEQ ID NOs: 2 and 4); G871A in SEQ ID NO: 1 (D291N in SEQ ID NOs: 2 and 4); C919G/T920A/G921C in SEQ ID NO: 1 (L307D in SEQ ID NOs: 2 and 4); G931A in SEQ ID NO: 1 (D311N in SEQ ID NOs: 2 and 4); A937G in SEQ ID NO: 1 (N313D in SEQ ID NOs: 2 and 4); G943A in SEQ ID NO: 1 (D315N in SEQ ID NOs: 2 and 4); and A964G in SEQ ID NO: 1 (N322D in SEQ ID NOs: 2 and 4). It is noted that the above-referenced nucleotide/amino acid substitutions can be present in any combination of subcombination in the nucleic acids and polypeptides of the presently disclosed subject matter. See for example, SEQ ID NOs: 3 and 4 of the Sequence Listing.

Relative degradation activities for the modified polypeptides were determined by measuring the reduction in packed cell volume (PCV) measured for each mutant when treated with enzyme mutants, and the results are presented herein below in Table 2:

TABLE 2
Change in PCV of Various CAases
Relative to Wild-type (WT) Using
Purified Colanic Acid as a Substrate

| | |
|---|---|
| WT | 1.0 |
| D287N | 0.22 |
| D291N | 0.21 |
| D311N | 0.3 |
| N313D | 0.93 |
| D315N | 1.4 |
| L307D | 1.9 |
| N284D | 2.5 |
| N322D | 3.1 |

The “wild type” polypeptide sequence of SEQ ID NO: 2 is from a Salmonella phage. The above-presented “wild-type” is the protein sequence. The above-presented “wild type” nucleotide sequence of SEQ ID NO: 1 is a codon-optimized version to improve recombinant protein expression in E. coli. In other words, the representative nucleotide sequence of SEQ ID NO: 1 is synthetic, although the amino acid sequence of SEQ ID NO: 2 is a wild-type Salmonella phage polypeptide sequence.

In some embodiments, the polypeptides are provided as part of a pharmaceutical composition.

II.D. Pharmaceutical Compositions

In some embodiments, the compositions of the presently disclosed subject matter are provided as part of a pharmaceutical composition. As used herein, the term “pharmaceutical composition” refers to a composition comprising at least one active ingredient (e.g., an inhibitor of the presently disclosed subject matter), whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

In some embodiments, a pharmaceutical composition of the presently disclosed subject matter comprises, consists essentially of, or consists of at least one active ingredient (e.g., an inhibitor of the presently disclosed subject matter) and a pharmaceutically acceptable diluent and/or excipient. As used herein, the term “pharmaceutically acceptable” refers to physiologically tolerable, for either human or veterinary application. Similarly, “pharmaceutical compositions” include formulations for human and veterinary use. The term “pharmaceutically acceptable carrier” also refers to a chemical composition with which an appropriate compound or derivative can be combined and which, following the combination, can be used to administer the appropriate compound to a subject. In some embodiments, a pharmaceutically acceptable diluent and/or excipient is pharmaceutically acceptable for use in a human.

In some embodiments, the pharmaceutical compositions of the presently disclosed subject matter are for use in inhibiting the growth of a microbe or a microbial biofilm on a surface and/or for inhibiting the growth of microbe on and/or in a subject.

The pharmaceutical compositions of the presently disclosed subject matter can in some embodiments consist of the active ingredient alone (e.g., the CAase polypeptide or fragment thereof of the presently disclosed subject matter), in a form suitable for administration to a subject, or the pharmaceutical composition can in some embodiments comprise or consist essentially of the active ingredient and one or more pharmaceutically acceptable carriers, one or more additional ingredients, or some combination of these. The active ingredient can be present in the pharmaceutical composition in the form of a physiologically acceptable ester or salt, such as in combination with a physiologically acceptable cation or anion, as is well known in the art.

As used herein, the term “physiologically acceptable” ester or salt refers to an ester or salt form of the active ingredient which is compatible with any other ingredients of the pharmaceutical composition, which is not deleterious to the subject to which the composition is to be administered.

The formulations of the pharmaceutical compositions described herein can be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts.

II.E. Formulations

The compositions of the presently disclosed subject matter thus comprise in some embodiments a composition that includes a carrier, particularly a pharmaceutically acceptable carrier, such as but not limited to a carrier pharmaceutically acceptable in humans. Any suitable pharmaceutical formulation can be used to prepare the compositions for administration to a subject.

For example, suitable formulations can include aqueous and non-aqueous sterile injection solutions that can contain anti-oxidants, buffers, bacteriostatics, bactericidal antibiotics, and solutes that render the formulation isotonic with the bodily fluids of the intended recipient.

It should be understood that in addition to the ingredients particularly mentioned above the formulations of the presently disclosed subject matter can include other agents conventional in the art with regard to the type of formulation in question. For example, sterile pyrogen-free aqueous and non-aqueous solutions can be used.

The therapeutic regimens and compositions of the presently disclosed subject matter can be used with additional adjuvants or biological response modifiers including, but not limited to, cytokines and other immunomodulating compounds.

Controlled- or sustained-release formulations of a pharmaceutical composition of the presently disclosed subject matter can be made using conventional technology. A formulation of a pharmaceutical composition of the invention suitable for oral administration can be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

Liquid formulations of a pharmaceutical composition of the presently disclosed subject matter which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin.

Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methy lcellulose, hydroxypropylmethylcellulose.

Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively).

Known emulsifying agents include, but are not limited to, lecithin and acacia. Known preservatives include, but are not limited to, methyl, ethyl, or n-propyl parahydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil in water emulsion or a water-in-oil emulsion.

The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the presently disclosed subject matter may also be prepared, packaged, or sold in a formulation suitable for parenteral administration, including but not limited to intraocular injection.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butane dial, for example.

Other acceptable diluents and solvents include, but are not limited to, Ringer's solution, isotonic sodium chloride solution, and fixed oils such as synthetic mono or di-glycerides. Other parentally-administrable formulations which are useful include those which comprise the active ingredient in microcrystalline form, in a liposomal preparation, or as a component of a biodegradable polymer systems.

Compositions for sustained release or implantation may comprise pharmaceutically acceptable polymeric or hydrophobic materials such as an emulsion, an ion exchange resin, a sparingly soluble polymer, or a sparingly soluble salt. Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, can in some embodiments have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed. (1985) Remington's Pharmaceutical Sciences, Mack Publishing Co., Easton, Pa., United States of America, which is incorporated herein by reference in itsz entirety.

II.F Administration

With regard to administering a composition of the presently disclosed subject matter, methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, subcutaneous administration, intravitreous administration, including via intravitreous sustained drug delivery device, intracameral (into anterior chamber) administration, suprachoroidal injection, subretinal administration, subconjunctival injection, sub-tenon administration, peribulbar administration, transscleral drug delivery, intraocular injection, intravenous injection, intraparenchymal/intracranial injection, intra-articular injection, retrograde ureteral infusion, intrauterine injection, intratesticular tubule injection, intrathecal injection, intraventricular (e.g., inside cerebral ventricles) administration, administration via topical eye drops, and the like. Administration can be continuous or intermittent. In some embodiments, a preparation can be administered therapeutically; that is, administered to treat an existing disease or condition. In some embodiments, a preparation can be administered prophylactically; that is, administered for prevention of a disease, disorder, or condition.

II.G. Dose

An effective dose of a composition of the presently disclosed subject matter is administered to a subject in need thereof. A “treatment effective amount” or a “therapeutic amount” is an amount of a therapeutic composition sufficient to produce a measurable response (e.g., a biologically or clinically relevant response in a subject being treated). Actual dosage levels of active ingredients in the compositions of the presently disclosed subject matter can be varied so as to administer an amount of the active compound(s) that is effective to achieve the desired therapeutic response for a particular subject. The selected dosage level will depend upon the activity of the therapeutic composition, the route of administration, combination with other drugs or treatments, the severity of the condition being treated, and the condition and prior medical history of the subject being treated. However, it is within the skill of the art to start doses of the compound at levels lower than required to achieve the desired therapeutic effect and to gradually increase the dosage until the desired effect is achieved. The potency of a composition can vary, and therefore a “treatment effective amount” can vary. However, using the assay methods described herein, one skilled in the art can readily assess the potency and efficacy of a candidate compound of the presently disclosed subject matter and adjust the therapeutic regimen accordingly.

For administration of a therapeutic composition as disclosed herein, conventional methods of extrapolating human dosage based on doses administered to a murine animal model can be carried out using the conversion factor for converting the mouse dosage to human dosage: Dose Human per kg=Dose Mouse per kg×12 (Freireich et al., 1966). Doses can also be given in milligrams per square meter of body surface area because this method rather than body weight achieves a good correlation to certain metabolic and excretionary functions. Moreover, body surface area can be used as a common denominator for drug dosage in adults and children as well as in different animal species (see Freireich et al., 1966). Briefly, to express a mg/kg dose in any given species as the equivalent mg/sq m dose, multiply the dose by the appropriate km factor. In an adult human, 100 mg/kg is equivalent to 100 mg/kg×37 kg/sq m=3700 mg/m².

After review of the disclosure of the presently disclosed subject matter presented herein, one of ordinary skill in the art can tailor the dosages to an individual subject, taking into account the particular formulation, method of administration to be used with the composition, and particular disease treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

### III. Methods of Use of the Polypeptides of the Presently Disclosed Subject Matter

In some embodiments, a method for inhibiting the growth of a microbe or a microbial biofilm on a surface, or disrupting a microbial biofilm on a surface, is provided. In some embodiments, the method comprises contacting the surface with an effective amount of an antimicrobial composition of the presently disclosed subject matter. In some embodiments, the surface is a surface of an agricultural product or food handling surface. In some embodiments, the surface is a surface of a medical device. In some embodiments, the surface is a surface on or in a subject.

In some embodiments, the surface is contaminated with a microbe, such as a bacterium, or a biofilm formed by the microbe (e.g., a bacterial biofilm). In some embodiments, the surface was exposed to a microbe, such as a bacterium; in yet another embodiment, the surface will be exposed to a microbe, such as a bacterium; in a further embodiment, the surface is at risk of being exposed to a microbe, such as a bacterium, or having a biofilm formed by the microbe (e.g., a bacterial biofilm) develop.

In some embodiments, a method for inhibiting the growth of microbe on, or in, an agricultural product is provided. In some embodiments, the method comprises administering an antimicrobial composition in accordance with the presently disclosed subject matter to the agricultural product. In some embodiments, the microbe is a pathogenic bacterium, such as but not limited to E. coli, Salmonella, Pseudomonas, Listeria, and/or Stenotrophomonas.

In some embodiments, a method for inhibiting the growth of microbe on, or in, a subject is provided. In some embodiments, the method comprises administering an antimicrobial composition in accordance with the presently disclosed subject matter to the subject. In some embodiments, the microbe is a pathogenic bacterium, such as but not limited to E. coli, Salmonella, Pseudomonas, Listeria, and/or Stenotrophomonas. In some embodiments, the antimicrobial composition comprises, consists essentially of, or consists of a polypeptide comprising, consisting essentially of, or consisting of an amino acid encoded by SEQ ID NO: 1 or SEQ ID NO: 3, such as but not limited to SEQ ID NO: 2 or SEQ ID NO: 4.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

With reference to the following Examples and without further description, it is believed that one of ordinary skill in the art can, using the preceding description and the following illustrative examples, make, utilize, and/or practice the presently disclosed and claimed subject matter. Therefore, the Examples should be construed to encompass any and all variations which become evident as a result of the teaching provided herein.

### Materials and Methods for the EXAMPLES

Enzyme Expression.

The general expression plasmids and methods for enzyme overexpression utilized in this work have been previously described in detail (MacDonald & Berger, 2014). In brief, the CAase gene was subcloned into a pET28a plasmid and transformed into E. coli BL21 cells by electroporation. Kanamycin-selective plates (50 μg/mL working concentration) were used to isolate individual colonies; these colonies were then inoculated in 10 mL Luria Bertani (LB) cultures containing kanamycin, and grown overnight at 37° C. in a shaking incubator (Innova R26 at 200 rpm). Cells from saturated cultures were then transferred to 100 mL of fresh LB media containing kanamycin and grown at 37° C. with shaking at 200 rpm for 1 hour, such that the cell density measured at 600 nm reached 0.6. To induce protein production, IPTG was added to the 100 mL culture at a working concentration of 1 mM, and the growth temperature changed to 20° C. After 16 hours of growth at 20° C. with 200 rpm agitation, cells were harvested by centrifugation at 3000 rpm for 10 minutes.

Enzyme Purification.

Cell pellets from 100 mL of induced culture were resuspended in 40 mL of lysis buffer (100 mm HEPES, 500 mm NaCl, 10% w/v glycerol, 10 mm imidazole) and then sonicated (Misonix 3000 Ultrasonic Cell Disruptor, 15 W, 20 min process time, 20 s on/20 s off pulses) in order to lyse the cells while in an ice bath. The 40 mL lysis mixture was centrifuged at 10000 rpm for 10 min and the soluble supernatant containing enzyme was collected. Centrifugation and disposal of insoluble material was repeated three times.

The enzyme was purified using immobilized metal ion affinity chromatography (IMAC) with 15 mL of Profinity resin, as previously described (Eckersley & Berger, 2018). In summary, one column volume of 0.2 M nickel chloride solution was added to charge the column, followed by three column volumes of deionized water and one column volume of lysis buffer. The cell lysate was then added to the column, allowed to mix gently for 10 min, and then washed with increasing concentrations (10-500 mM) of imidazole, primarily using imidazole concentrations of 250 mM and 500 mM to elute the protein. Eluent was collected in 5 mL fractions and SDS-PAGE was used to confirm purification and purity of final enzyme product. Protein samples (20 μL) were mixed with 5 μL of SDS-PAGE running buffer and heated for 10 min at 90° C. to denature proteins before loading 15 μL aliquots onto a 4% stacking, 12% separating acrylamide gel with IVIES running buffer. Precision Plus Protein All Blue Standard (Bio-Rad) was used as a molecular weight standard. The gel was run at 100 V for 15 min and then at 175 V for 40 min. The gel was then stained with Coomassie Blue stain (1 g Coomassie Brilliant Blue (Bio-Rad), 1:4:5 acetic acid, methanol, double-distilled water) for 2 h and then destained with a solution of 1:2:7 acetic acid, methanol and double-distilled water. Collected column fractions that contained purified protein were dialyzed for 24 h at 4° C. with a 7000 MWC ThermoFisher Snakeskin dialysis membrane in 4 L of 75 mM pH 8 phosphate buffer, and lyophilized for long-term storage or used immediately. Enzyme concentration was determined by measuring absorbance at 280 nm, using a calculated extinction coefficient of 121990 M⁻¹ cm⁻¹ based on primary sequence.

Bacteria Growth and Harvest.

To test enzyme effectiveness on foodborne bacteria, E. coli ATCC 25922, E. coli O157:H7 (ATCC 4388), Salmonella typhimurium (ATCC 13311), and Listeria monocytogenes (ATCC 19117) were used as model bacteria in this study, obtained from the USDA (Kimberly Cook, USDA-ARS-FAESR, Bowling Green, Ky.). E. coli O157:H7, Salmonella typhimurium, and Listeria monocytogenes are pathogens that have been implicated in foodborne illness outbreaks associated with fresh produce (Bennett et al., 2015; Callejón et al., 2015; Sharapov et al., 2016). E. coli 25922 is a non-pathogen surrogate strain that has been identified and used to model pathogens in food safety environments (Kim & Harrison, 2009). E. coli and Salmonella cells were cultured in Luria-Bertani (LB) broth (Fisher Scientific, Fair Lawn, N.J.) and Listeria cells were cultured in tryptic soy broth (TSB) at 37° C. overnight. For biofilm assays, cells from the overnight culture were diluted 1:100 in 2 mL of minimal media and grown for 48 hr at 37° C. under static conditions. Minimal media was composed of one half standard broth (LB for E. coli and Salmonella, TSB for Listeria) and one half M9 media, which was created using 6 mg/mL Na₂HPO₄, 3 mg/mL, KH₂PO₄, 0.5 mg/mL NaCl, and 1 mg/mL NH4Cl, supplemented with 1% glucose, 2 mM MgSO₄, and 0.1 mM CaCl₂ in deionized water (Anonymous, 2010).

For flow cell detachment experiments, E. coli O157:H7 cells from overnight culture were transferred to 200 mL fresh LB media and harvested at the mid-exponential cell growth phase by centrifugation at 3000 rpm for 10 minutes and resuspension in 10 mM KCl three times (Haznedaroglu et al., 2009). This simple salt solution chemistry was chosen to represent an environmentally relevant ionic strength within the realm of possibility for surface and groundwater, and also to maximize observable attachment, as shown by previously reported trends in microbial adhesion to the epicuticle and other solid surfaces (Rapicavoli et al., 2015). Bacterial cell suspensions were adjusted to a final optical density of 0.2 at 600 nm, corresponding to approximately 10⁸ cells/mL

Biofilm Assays.

Biofilm growth experiments were conducted using sterile 24-well polystyrene plates (Corning Inc., Corning, N.Y.). Plates were prepared in duplicate, wrapped in alumni foil to minimize evaporation, and incubated at 32° C. for 48 hr. Each plate included four wells of uninoculated M9 minimal media as control wells. After the 48 hr incubation period, 100 μL of 1% crystal violet in 95% ethanol was added to each well and allowed to incubate at room temperature for 20 min. The medium was then removed from wells and microtiter plate wells were washed five times with sterile distilled water to remove loosely associated bacteria. At this point, biofilms were visible as purple rings formed on the side of each well at the air-liquid interface and plates were air dried at room temperature for 45 min. Biofilm production was quantified by adding 2 mL of 20% acetone/80% ethanol to destain each of the wells and allowing to mix gently for 20 min. The absorbance was measured at 600 nm to quantify the crystal violet present in the destaining solution. Each assay was performed at least three times and the averages and standard deviations were calculated for all repetitions.

For biofilm inhibition assays, 0.1 mg/mL CAase was added to each well at the beginning of the 48 hour incubation period. For biofilm removal assays, minimal media was removed from each well after 48 hour and replaced with 2 mL of 0.1 mg/mL CAase in 10 mM KCl or plain 10 mM KCl for controls. Plates were incubated at room temperature for 20 minutes before adding 100 μL of 1% crystal violet in 95% ethanol to begin the staining assay described above.

Parallel-Plate Flow Cell.

Bacterial detachment experiments were conducted in a parallel plate flow chamber (GlycoTech, Gaithersburg, Md.) positioned on an inverted fluorescent microscope (BX-52, Olympus) to allow for direct of cells attaching and detaching on the surface (McClaine & Ford, 2002; Chen et al., 2009; Kinsinger et al., 2017). The inner dimension of the chamber was 6 cm×1 cm×0.08 cm and was composed of a PLEXIGLAS® block, mounted to a microscope slide (supporting isolated spinach epicuticle layer on polycarbonate) by a flexible silicone elastomer gasket that was sealed by vacuum grease. The spinach leaf surface was prepared using a freeze-imbedding technique to separate the wax epicuticle layer from the rest of the leaf and transfer to a polycarbonate slide, as previously described (Kinsinger et al., 2017).

The influent entered the flow chamber from a capillary tube that was connected to a syringe, which was controlled by a syringe pump at a flow rate of 0.1 mL/min, which simulated expected surface conditions in a gentle leafy greens washing process (Huang & Nitin, 2017). The bacteria were imaged by a 40× long working distance objective (UPlanFl, Olympus), and connected to a computer running SimplePCI to record images with a digital camera (Demo Retiga EXI Monochrome, Qlmaging). Cells were allowed to attach over a 30 min period, followed by a 30 min rinse with 10 mM KCl solution containing 0, 250, or 1000 ppb CAase enzyme. In order to determine the kinetics of cell detachment, images were recorded every 30 s and enumeration of cells was determined by comparison of successive images.

Mass Transfer Rate Coefficients.

During all rinsing experiments, bacterial detachment was negligible beyond a certain time point, resulting in a plateau in the number of remaining, attached bacteria. Detachment mass transfer rate coefficients were calculated using the enumeration of observed cells up the plateau point, using MATLAB (R2015a, Mathworks, Natick, Mass.) to process collected images. The number of bacterial cells removed from the epicuticle surface was plotted versus time, and bacterial flux, J, was calculated by dividing the slope of the line by the microscope viewing area (230 mm×170 mm). The mass transfer rate coefficient for the bacteria, k, is calculated using the bacterial flux (number of cells per area per time), and the bulk cell concentration (number of cells per mL), C₀, via (Chowdhury et al., 2012; Elimelech et al., 2013):

$$k = \frac{J}{{C}_{0}}$$

In addition to mass transfer rate coefficients, total number of cells removed from the surface, normalized by the number of cells present at the beginning of the rinse phase, are reported. Each experiment was performed in triplicate using E. coli O157:H7.

Relative Hydrophobicity.

Hydrophobicity analysis of the bacteria was done by using the microbial adhesion to hydrocarbon (MATH) test that has previously described in detail (Rosenberg et al., 1980; Pembrey et al., 1999). In brief, bacteria were first diluted to an optical density of 0.2 at a wavelength of 600 nm in 10 mM KCl. One mL of n-dodecane (Fisher Scientific) was added to three assays of 4 mL of diluted bacteria suspension and each of the assays were vortexed for 3 min. Partitioning of cells between n-dodecane and the electrolyte solution was then determined by measuring absorbance after 45 min. Relative hydrophobicity was calculated as the percent of total cells partitioned into the hydrocarbon layer.

Electron Microscopy.

E. coli PHL624 cells grown under conditions to favor biofilm formation were collected, resuspended in minimal growth medium used to generate biofilm, and then placed on a 400 mesh copper grid containing holey carbon coated with an ultrathin carbon film. 2% ammonium molybdate was used as a counterstain for contrast imaging, and images of individual cells were taken using a benchtop EM (LVEM, Zeiss).

Statistical Analysis.

At least three independent repetitions were performed for characterization and all experiments, including a fresh cell culture for each trial. To test for differences between enzyme treatment and control conditions in all experiments listed above, a t-test was conducted to determine statistically significant differences for confidence intervals of 95% and 99% (p<0.05 and p<0.01, respectively).

### Example 1

### Enzyme Production

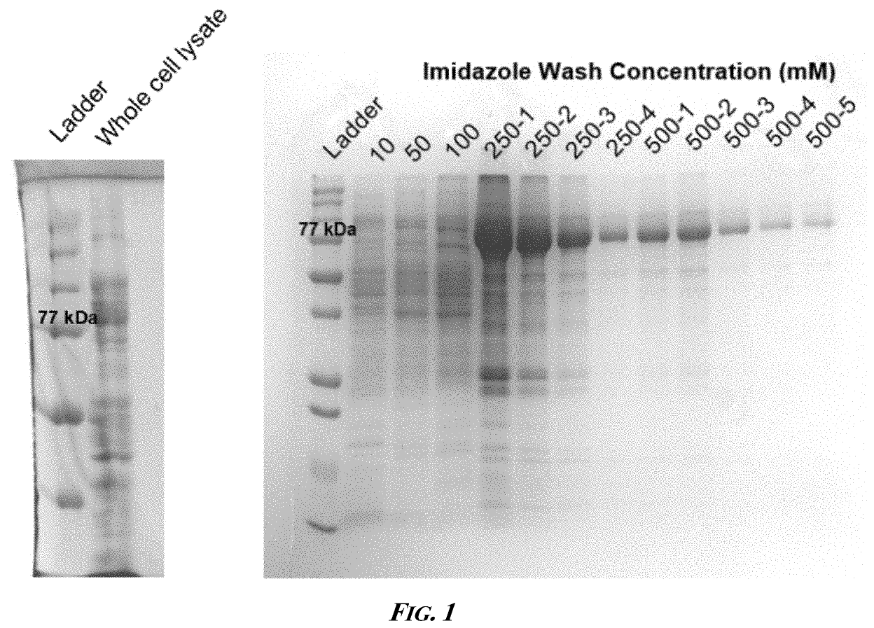

Expression of the recombinant, hexahistidine-tagged enzyme from BL21 cells indicated high-yields after IPTG induction as well as significant recovery after cell lysis and purification using standard IMAC affinity chromatography. As shown in the SDS-PAGE gel depicted in FIG. 1, a prominent band at 77 kDa was observed during purification; this size corresponded with the predicted molecular weight of CAase. Purified enzyme was collected in the 250 and 500 mM imidazole washes and dialyzed against pH 8 phosphate buffer to remove residual salts and impurities. The yields of purified enzyme were estimated to be 0.1 g enzyme/L culture, with the majority of the expressed protein recovered via IMAC affinity chromatography based on band intensities measured from SDS-PAGE.

### Example 2

### Inhibition of Biofilm Growth

To assess the ability of the enzyme to inhibit biofilm formation, E. coli 25922, E. coli O157:H7, Salmonella typhimurium, and Listeria monocytogenes were used as model, agriculturally-relevant bacteria. Biofilm formation of these strains have been previously investigated as a function of nutrient conditions, and collectively provided a representative set of gram-negative and gram-positive pathogens, as well as a quality control non-pathogen surrogate (Cook et al., 2017).

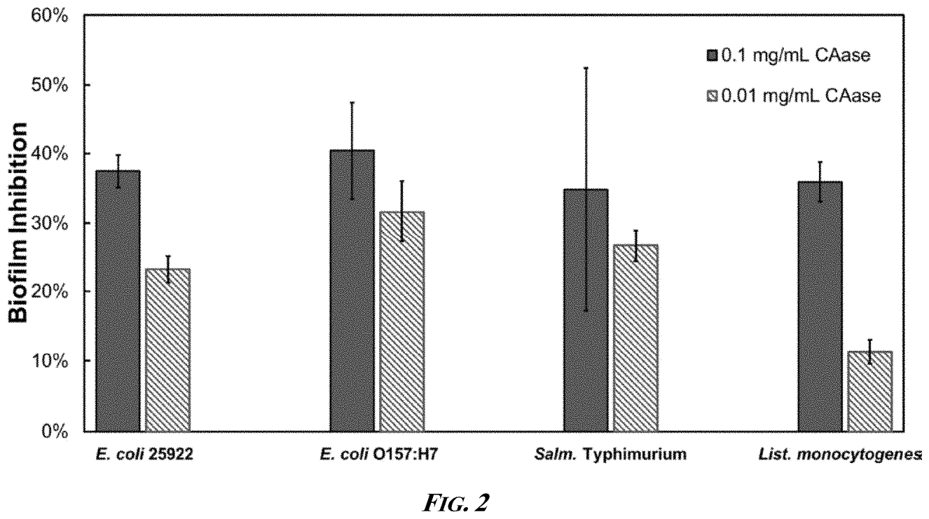

The addition of 0.1 mg/mL CAase resulted in significant inhibition of biofilm formation for all four cell types in terms of comparing relative levels of crystal violet staining of biofilm polysaccharides before and after treatment (see FIG. 2). Biofilm formation was reduced by 37.4±2.4% for E. coli 25922, 40.4±7.0% for E. coli O157:H7, 34.8±17.6% for Salmonella typhimurium, and 35.9±2.8% for Listeria monocytogenes. A lower enzyme concentration (0.01 mg/mL) was also effective, reducing biofilm formation by 23.2±2.4%, 31.6±4.4%, 26.6±2.3%, and 11.3±1.7% for E. coli 25922, E. coli O157:H7, Salmonella typhimurium, and Listeria monocytogenes, respectively. Previous studies have demonstrated reduced removal of mature biofilms, as well as reduced biofilm formation when treating with enzymes for specific bacterial species (Boyd & Chakrabarty, 1994; Izano et al., 2007). Interestingly, broad-range biofilm inhibition for multiple pathogens from a single enzyme was observed, which had not been described previously for other biofilm-degrading enzymes.

### Example 3

### Removal of Mature Biofilms

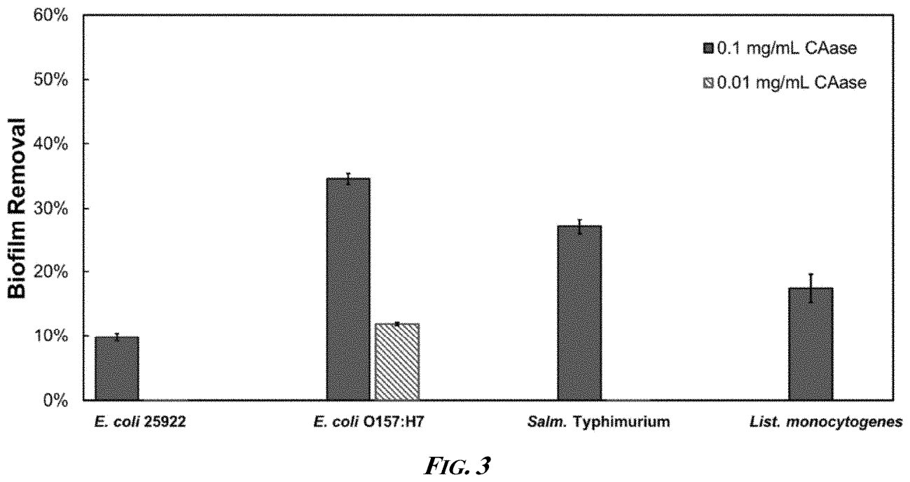

Biofilm removal with 0.1 mg/mL CAase was also compared to rinsing with a simple 10 mM KCl salt solution to mimic rinsing with tap water. The results (FIG. 3) demonstrated that CAase could also be effective in enhancing the disruption of established biofilms on surfaces. For the non-pathogen E. coli 25922, biofilm removal was enhanced by 9.8±0.6% with the presence of 0.1 mg/mL CAase. For the pathogen biofilms, 34.6±0.9%, 27±1.2% and 17.4±2.2% greater biofilm removal was observed for E. coli O157:H7, Salmonella typhimurium, and Listeria monocytogenes, respectively. At 0.01 mg/mL, CAase was largely ineffective at enhancing biofilm removal, with the exception of E. coli O157:H7 for which 11.9±0.2% less biofilm was present after enzyme treatment. Once removed from the biofilm matrix, planktonic cells are anticipated to be more susceptible to disinfectants, even at relatively low concentrations (Meireles et al., 2017); this is of particular interest in food safety applications, where removal or weakening of biofilms without physical or mechanical intervention remains a challenge (Gibson et al., 1999). Thus, these results demonstrated that added enzyme was effective in enhancing the removal of biofilms as well as prevention of biofilm formation as compared to mechanical disruption and washing using standard saline solution.

### Example 4

### Detachment from Spinach Leaf Surfaces

During the initial stages of biofilm formation, a transition from reversible to irreversible bacterial attachment occurs. These cells remain adhered to surfaces and produce the extracellular matrix that makes up a biofilm (Palmer et al., 2007; Blaschek et al., 2015; Álvarez-Ordóñez & Briandet, 2016). The initial reversible and irreversible bacterial attachment phases are unique from the rest of the biofilm formation process, as van der Waals forces, electrostatic forces, and hydrophobic interactions between cells and substrates are expected to play important roles (Palmer et al., 2007; Van Houdt & Michiels, 2010). To investigate the impact added enzyme has on initial attachment, the initial attachment phase was directly observed with light microscopy using a parallel plate flow cell to image attachment of E. coli O157:H7 cells onto a modal spinach leaf surface; this approach has been described previously for studying effects of bleach on bacterial attachment to spinach leaf surfaces. Given that E. coli O157:H7 cells were observed to have the most significant reductions in mature biofilm after a short period of enzyme treatment (FIG. 3), this cell type was chosen for the investigation of initial cell attachment. Based on previous work that utilized surface roughness data and COMSOL modeling to predict minimum disinfectant concentrations on the leaf surface, CAase concentrations three order of magnitude below the relevant bulk concentration were used in the flow cell (250 ppb and 1000 ppb for 250 ppm (0.25 mg/mL) and 1000 ppm (1 mg/mL), respectively; Kinsinger et al., 2017).

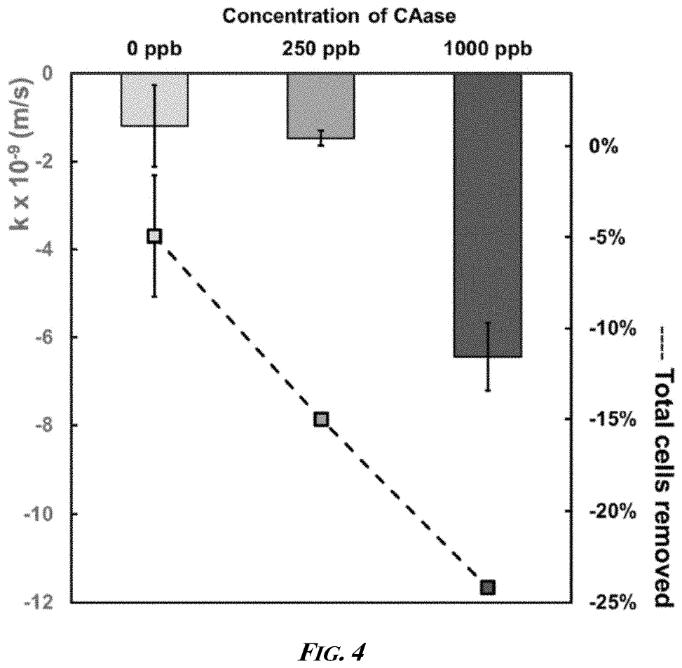

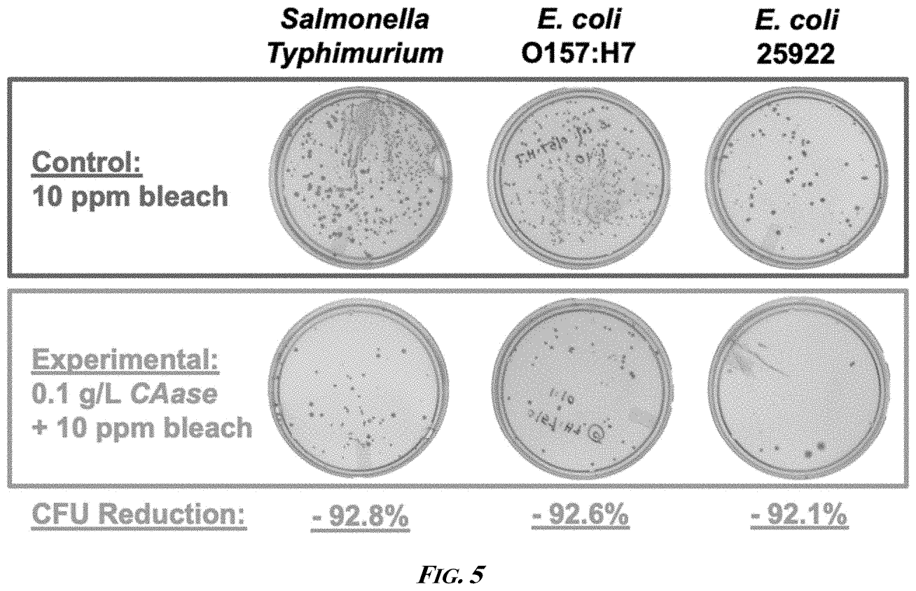

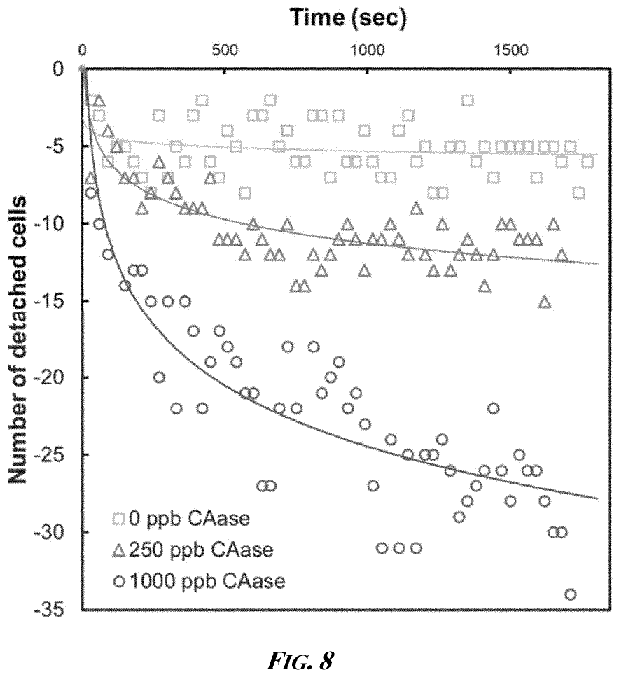

Detachment mass transfer rate coefficients for E. coli O157:H7 cells did not change significantly with added enzyme, increasing from −1.19±0.92×10⁻⁹ m/s with no CAase to −1.47±0.17×10⁻⁹ m/s with 250 ppb CAase in the rinse solution (see FIGS. 4 and 5 and Table 3). However, total detached cells increased from 5% to 15%, indicating that the time over which detachment is observed was greater with the enzyme rinse versus 10 mM KCl control solution without enzyme (FIG. 8). Detachment rates with 1000 ppb enzyme were more than five times greater than the DI water rinse, increasing from −1.19±0.92×10⁻⁹ m/s to −6.44±0.77×10⁻⁹ m/s. Additionally, 24% of the total number of cells were removed from the surface with 1000 ppb of CAase over the 30-minute rinse. Thus, these results indicate that added enzyme at equivalent concentrations used for chemical cleaning treatments causes uniform increases in the total amount of time over which bacterial release from the surface occurs relative to saline rinse, thereby leading to an overall increase in total bacterial removal with enzyme treatment. Furthermore, increasing the enzyme concentration to 1000 ppb can increase both the rate of detachment as well as the total time over which detachment occurs, leading to substantially greater total bacterial removal from the surface.

Work with E. coli O157:H7 cells observed comparable detachment rate coefficients to 1000 ppb CAase with 10 ppb sodium hypochlorite (bleach), which is the maximum allowable sodium hypochlorite concentration allowed in postharvest handling of organic produce (Suslow, 2000; Kinsinger et al., 2017). Additionally, 1000 ppb CAase resulted in steady bacterial detachment observed throughout the 30-minute experiment (FIG. 8), while the detachment phase with various bleach concentrations never exceeded 16 minutes (Kinsinger et al., 2017). These results offer additional support that enzymes such as CAase may provide a useful alternative or complement to traditional processing disinfectants with potentially greater extent of total bacteria removed during rinsing. Thus, CAase has the ability to significantly increase bacterial removal rates under continuous, dynamic washing conditions for an extended period of time as compared to bleach, rendering them susceptible to other disinfectants used in solution once released.

TABLE 3
Colony-forming Unites (CFUs) Observed under
Serial Dilutions of Treated Spinach Leaves

| | | | | | |
|---|---|---|---|---|---|
| | | | 10 ppm | | |
| | | | Bleach + | | |
| | 10 ppm | Total | 0.1 mg/ML | Total | % |
| Dilution | Bleach | CFU | CAase | CFU | Reduction |

E.coli 25922

| | | | | | |
|---|---|---|---|---|---|
| 1:1 | — | 57133 | — | 4500 | 92.12 |
| 1:10 | 434 | 43400 | 70 | 7000 | 83.87 |
| 1:100 | 98 | 98000 | 2 | 2000 | 97.96 |
| 1:1000 | 3 | 30000 | 0 | 0 | 100.00 |

E.coli O157:H7

| | | | | | |
|---|---|---|---|---|---|
| 1:1 | — | 35633 | — | 2650 | 92.56 |
| 1:10 | 429 | 42900 | 43 | 4300 | 89.98 |
| 1:100 | 14 | 14000 | 1 | 1000 | 92.86 |
| 1:1000 | 5 | 50000 | 0 | 0 | 100.00 |

Salmonella typhimurium

| | | | | | |
|---|---|---|---|---|---|
| 1:1 | — | 32833 | — | 2350 | 92.84 |
| 1:10 | 85 | 8500 | 17 | 1700 | 80.00 |
| 1:100 | 10 | 10000 | 3 | 3000 | 70.00 |
| 1:1000 | 8 | 80000 | 0 | 0 | 100.00 |

### Example 5

### Mechanisms of Enzyme Action

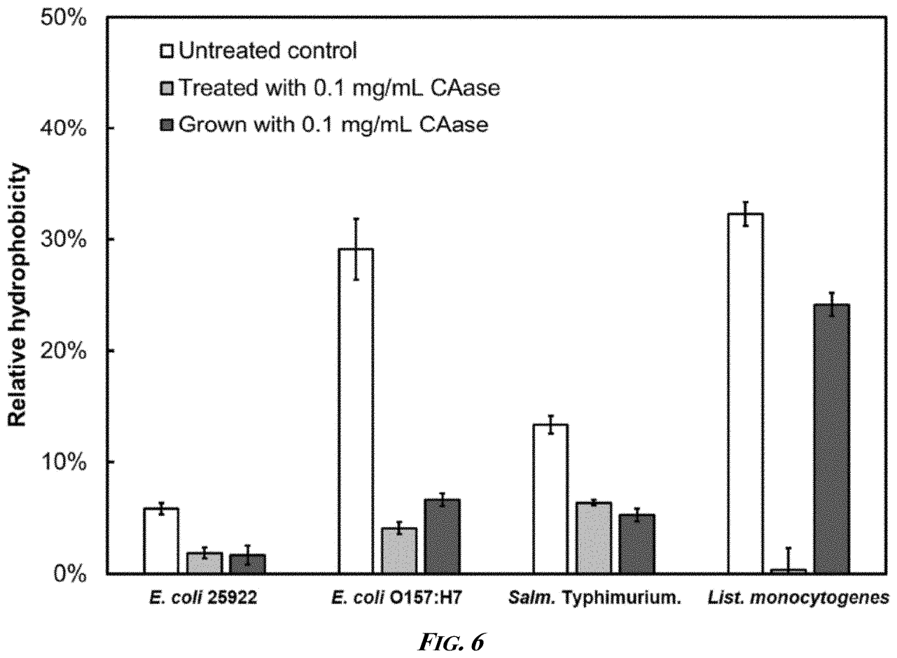

To assess the impact of CAase on the bacterial cell wall and extracellular environment, the cell surface structure was analyzed indirectly through relative hydrophobicity and directly through electron microscopy. Relative hydrophobicity of cells refers to the percentage of cells remaining in a 10 mM KCl solution versus partitioning into a hydrocarbon layer through the microbial adhesion to hydrocarbons (MATH) assay. Surface modification of bacteria, including changes in surface polysaccharides, is reflected in changes in relative hydrophobicity measured via the MATH test. Relative hydrophobicity was significantly reduced for all four strains after treatment with 0.1 mg/mL CAase for 20 min in suspension (FIG. 6). Listeria monocytogenes showed the largest decrease (32.3±1.0% to 0.3±1.9% for the control and treated samples, respectively), followed by the reduction of E. coli O157:H7 from 29.1±2.7% to 4.0±0.5%, Salmonella typhimurium from 13.3±0.8% to 6.3±0.2%, and E. coli 25922 from 5.7±0.5% to 1.8±0.5%. For these short-term exposure treatment assays intended to simulate biofilm removal scenarios, the enzyme remained in solution with cells for the duration of the MATH assay (light gray bars in FIG. 6). To assess the impact of the long-term exposure and simulate biofilm inhibition scenarios, cells were also grown in the presence of 0.1 mg/mL CAase and separated from the enzyme before the MATH assay (dark gray bars in FIG. 6). For E. coli 25922, E. coli O157:H7, and Salmonella species, relative hydrophobicity did not significantly differ between these two scenarios. However, the relative hydrophobicity of Listeria cells appeared to recover (24.1±1.0% versus 32.3±1.0% for untreated control) after being grown with and separated from CAase.

Hydrophobic interactions are considered a major driving force for adhesion of bacteria cells to both biotic and abiotic surfaces (Hood & Zottola, 1995; Palmer et al., 2007). Previous studies using multiple strains of foodborne pathogens, including various E. coli, Salmonella, and Listeria stains, have demonstrated that reduced hydrophobicity plays a key role in reducing bacterial attachment to surfaces and ultimately biofilm formation (Walker et al., 2005; Di Bonaventura et al., 2008; Patel et al., 2011; Wang et al. 2013), although this effect can be highly dependent on the produce type. Specifically, changes in cell surface exopolysaccharides (EPS) and lipopolysaccharides (LPS) can contribute to measurable changes in cell surface hydrophobicity and observed attachment to food surfaces (Park & So, 2000; Zhao et al. 2015; Ebbensgaard et al., 2018). While the influence of extracellular polymers is debated, several studies have found that the presence and exposure of LPS can be correlated with increased cell surface hydrophilicity (Al-Tahhan et al. 2000; Park & So, 2000).

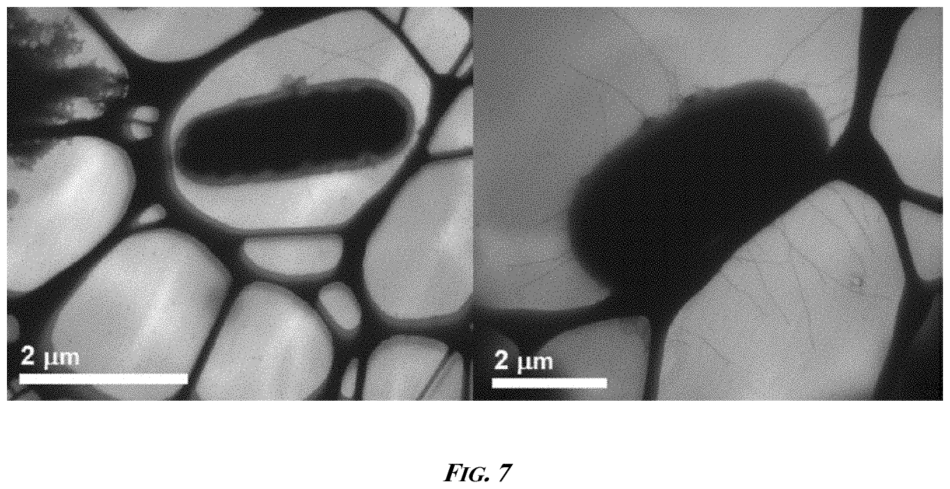

As set forth herein, it is possible that the enzyme degrades the outer polysaccharide regions, leaving inner hydrophilic cell surface structures that make up LPS exposed, resulting in relative hydrophobicity of less than 10% for almost every treatment scenario (Madigan et al., 1997; Walker et al., 2004). Electron microscopy (EM) of treated and untreated E. coli 25922 cells provide results consistent with a mechanism of polysaccharide degradation involving surface and extracellular polysaccharides. In FIG. 7, cells from the untreated control (left) appear with visibly intact cell walls, while cells exposed to CAase (right) appear shrunken with collapsed or missing cells walls and visible cell leakage. These EM images are consistent with those of other studies that have demonstrated the efficacy of various disinfectants to compromise and damage the cell surface (48-50). For example, Al-Hashimi and co-workers observed similar EM images of E. coli BL21 cells after co-treatment with ultrasound and ozone in water (Al-Hashimi et al., 2015). In the absences of surface and extracellular polysaccharides, bacterial cells are expected to be more vulnerable to collapse and cell death by changes in pH and temperature, as well as osmotic and oxidative stress, as has been demonstrated by EPS-deficient E. coli O157:H7 mutants (Chen et al., 2004).

The observed differences in effects of enzyme treatment between bacterial strains may be a function of differences in their respective mechanisms of attachment and biofilm composition. For example, cellulose and curli been demonstrated to be crucial components of the extracellular matrix that promote adhesion and biofilm formation in both E. coli and Salmonella strains (Madigan et al., 1997; Zogaj et al., 2001; Solano et al., 2002). However, Uhlich and co-workers demonstrated that curli is uncommon for pathogenic E. coli O157:H7 strain ATCC 43888, specifically (Uhlich et al., 2001). This may explain the observed consistent and prominent changes in E. coli O157:H7 biofilms and cellular attachment, versus the other bacteria employed in this study. Degradation of cellulose and other polysaccharides by the enzyme may disrupt biofilms and restrict adhesion, but curli and other proteins are not expected to be susceptible to enzymatic action.

Non-pathogenic E. coli 25922 is a common surrogate for biofilm assays in both agricultural and clinical testing. The strain produces significant amounts of EPS when grown in LB media (Solomon et al., 2005; Foppen et al., 2008; Mayton et al., 2019b). However, EPS production by E. coli 25922 has not been consistently correlated with significant biofilm formation in high- or low-nutrient conditions (Cook et al., 2017). The relatively high negative zeta potential and resulting electrostatic repulsion of E. coli 25922 cells has been attributed to this discrepancy. This characteristic may similarly minimize bacterial interactions with CAase and explain the smallest observed efficacy of enzymatic biofilm removal (Foppen et al., 2008; Mayton et al., 2019b). This is further exemplified by the minimum biofilm removal and change in cell surface hydrophobicity after enzyme treatment of E. coli 25922 cells, in comparison to larger changes in the other pathogenic strains used in this study.

In contrast to the E. coli strains, Salmonella typhimurium strain ATCC 13311 has been found to produce curli, but not cellulose on LB agar (Solomon et al., 2005; Cook et al., 2017). Others have demonstrated that flagella are the most important extracellular structure in Salmonella adherence to plant surfaces (Walsh et al., 2003; Mayton et al., 2019a). Still, significant biofilm inhibition is observed for Salmonella typhimurium in this study, which is comparable to the observed impact on E. coli species. This may be explained by the influence of low-nutrient growth conditions employed for biofilm formation assays, though literature on extracellular polysaccharide and protein production for Salmonella in similar conditions is limited. However, our previous work with these E. coli O157:H7 and Salmonella typhimurium strains demonstrated that while extracellular polysaccharide production was generally suppressed for E. coli in M9 minimal media, production was unchanged or increased in Salmonella cells (Mayton et al., 2019a). Therefore, more biofilm as substrate may be available for enzyme activity in minimal media, resulting in more significant impacts on biofilm formation.

The influence of CAase on the gram-positive species Listeria monocytogenes offers additional insights into potential mechanisms of activity on biofilms and individual bacterial cells. While many known biocidal enzymes are active against either gram-positive or gramnegative organisms, CAase was effective against biofilms of both types of pathogens. This implies that the enzyme acts on a common component of the biofilm matrix, such as extracellular polysaccharides. However, enzyme treatment is significantly less effective on inhibiting establishment of Listeria biofilms at the lower enzyme concentration (0.1 mg/mL), compared to the gram-negative organisms (FIG. 2). This implies that CAase activity is less efficient on the gram-positive cell type, which could be attributed to the thicker cell wall that offers greater resistance to degradation or to the lesser presence and availability of cell surface polysaccharides (Walsh et al., 2003; Misra et al., 2015). Additionally, Listeria cells are unique in their recovery of cell surface hydrophobicity after being grown with and then separated from the enzyme in solution. As displayed in FIG. 6, the hydrophobicity of the other bacteria species remains low after the removal of the enzyme from solution. Therefore, CAase must have the ability to physically associate with and modify the cell surface of gram-positive organisms to suppress hydrophobicity and biofilm formation to an extent, but the effect of enzyme activity is not significant enough to have a lasting impact on the cell surface after removing the enzyme from solution. This observation implies CAase may act on exopolysaccharide species, which are essential to biofilm formation and cell aggregation for many gram-negative species including L. monocytogenes, and minimal and less significant for gram-positive species. Additional research is required to further elucidate the specific mechanisms of enzyme activity, enzyme substrates as well potential physical interactions with the cell surface and biofilm matrix.

### Discussion of the EXAMPLES

To minimize risks to public health, strategies to prevent biofilm formation are arguably more efficient than controlling and removing mature biofilms (Eleftheriadou et al., 2017). Other proposed methods for inhibiting biofilm formation in the food industry include modification or treatment of surfaces to discourage bacterial attachment. For example, the potential of increasing surface roughness, hydrophilicity, and zeta potential, as well as the incorporation of antimicrobials like nano-silver, have been demonstrated (Jansen & Kohnen, 1995; Arnold & Bailey, 2000; Eleftheriadou et al., 2017). However, these approaches to preventing biofilm formation require industry transition and investment in new materials and processing equipment. Additionally, surface modification is often not a feasible or safe option for addressing bacterial adhesion to produce surfaces.

The observed differences in efficacy of the enzyme functionality between Salmonella and E. coli strains may be a function of differences in their respective mechanisms of attachment and biofilm composition. Cellulose and curli been shown to be crucial components of the extracellular matrix that promote adhesion and biofilm formation in both E. coli and Salmonella typhimurium (Zogaj et al., 2001; Solano et al., 2002; Castelijn et al., 2012). Degradation of cellulose and other polysaccharides by the enzyme may disrupt biofilms and restrict adhesion, but curli and other proteins are not susceptible to enzymatic action. Therefore, these results imply that proteins may dominate adhesion mechanisms for E. coli cells in these conditions. Previous studies have observed curli expression by various strains of E. coli O157:H7 in similar growth conditions; specifically, temperatures below 37° C. and in low salt medium (Kim et al., 2009; Saldana et al., 2009; Patel et al., 2010). Further, curli expression has been correlated with biofilm forming potential by E. coli O157:H7 (Ryu et al., 2004; Pawar et al., 2005), including strains isolated from a spinach-related outbreak in 2006 (Uhlich et al., 2008). Macarisin et al. found that curli were essential for attachment of E. coli O157:H7 to spinach leaf surfaces, while cellulose was considered dispensable (Macarisin et al., 2012). Alternatively, Solano et al. (2002) showed that cellulose played a critical role in biofilm formation by Salmonella enteritidis (Solano et al., 2002), which may render its biofilms more susceptible to enzyme treatment.

Overall, removal or weakening of biofilms without physical or mechanical intervention remains a challenge (Gibson et al., 1999). However, planktonic cells are significantly more susceptible to disinfectants, even at relatively low concentrations. These results are especially promising, as they demonstrate the enzyme's ability to disrupt the biofilms both during and after formation, leaving cells planktonic and potentially enhancing the efficacy of disinfectants.

Summarily, biofilm formation is one of the main causes of post-harvest pathogenic bacteria persistence on leafy green surfaces. These pathogens may lead to foodborne illnesses due to enhanced microbial resistance to common sanitizers, such as bleach. In accordance with the presently disclosed subject matter, a predicted glycosyl hydrolase was expressed, purified, and demonstrated to significantly inhibit biofilm formation and remove existing biofilms from a range of gram-positive and gram-negative bacteria. Furthermore, the results disclosed herein were consistent with an ability of the enzyme to enhance or replace chlorine in food processing applications. To produce the hydrolase enzyme, the protein was expressed recombinantly and purified from BL21 cells. Then, changes in biofilm growth by E. coli O157:H7, E. coli 25922, Salmonella typhimurium, and Listeria monocytogenes on polystyrene were up to 40% inhibited by the presence of 0.1 mg/mL of the enzyme, providing evidence that the hydrolase was able to effectively degrade the extracellular matrix that typically protects cells and supports attachment. The early stages of biofilm formation by E. coli O157:H7 cells on spinach leaf surfaces was directly observed in the parallel-plate flow cell. Detachment rate coefficients and total detached cells were significantly increased with the addition of 1000 ppb CAase to the rinse solution, which suggested that the enzyme was able to effectively reverse the foundational step in the biofilm formation process. Additionally, reductions in cell surface hydrophobicity and damaged cells observed through election microscopy after enzyme treatment shed some light on potential enzyme activity as a polysaccharide hydrolase.

### REFERENCES

All references listed below, as well as all other references cited in the instant disclosure, including but not limited to all patents, patent applications and publications thereof, scientific journal articles, and database entries (e.g., GENBANK® and UniProt biosequence database entries and all annotations available therein) are incorporated herein by reference in their entireties to the extent that they supplement, explain, provide a background for, or teach methodology, techniques, and/or compositions employed herein.
Al-Tahhan et al. (2000) Rhamnolipid-induced removal of lipopolysaccharide from Pseudomonas aeruginosa: Effect on cell surface properties and interaction with hydrophobic substrates. Applied and Environmental Microbiology, 66(8):3262-3268.
Altschul et al. (1990a) Basic local alignment search tool. J. Mol. Biol. 215:403-410.
Altschul et al. (1990b) Protein database searches for multiple alignments. Proc. Natl. Acad. Sci. USA. 87:14:5509-13.
Altschul et al. (1997) Gapped BLAST and PSI-BLAST: a new generation of protein database search programs. Nucleic Acids Res. 25:3389-3402.
Álvarez-Ordóñez & Briandet (2016) Editorial: Biofilms from a Food Microbiology Perspective: Structures, Functions, and Control Strategies. Front Microbiol 7.
Anonymous (2010) M9 minimal medium (standard). Cold Spring Harbor Protocols 2010:pdb.rec12295.
Arnold & Bailey (2000) Surface finishes on stainless steel reduce bacterial attachment and early biofilm formation: Scanning electron and atomic force microscopy study. Poultry Science, 79(12):1839-1845.
Bennett et al. (2015) Multistate foodborne disease outbreaks associated with raw tomatoes, U.S., 1990-2010: A recurring public health problem. Epidemiology and Infection, 143(7):1352-1359.
Blaschek et al. (2015) Biofilms in the Food Environment. Blackwell Publishing Professional, Ames, Iowa.
Bodanszky & Bodanszky (1984) in The Practice of Peptide Synthesis, Springer-Verlag, New York, N.Y.
Boyd & Chakrabarty (1994) Role of alginate lyase in cell detachment of Pseudomonas aeruginosa. Appl Environ Microbiol 60(7):2355-2359.
Callejón et al. (2015) Reported foodborne outbreaks due to fresh produce in the United States and European Union: Trends and causes. Foodborne Pathogens and Disease, 12(1):32-38.
Castelijn et al. (2012) Diversity in biofilm formation and production of curli fimbriae and cellulose of Salmonella typhimurium strains of different origin in high and low nutrient medium. Biofouling, 28(1):51-63.
Chen et al. (2004) Protective effect of exopolysaccharide colanic acid of Escherichia coli O157:H7 to osmotic and oxidative stress. Int J Food Microbiol 93:281-286.
Chen et al. (2009) Initial bacterial deposition on bare and zeolite-coated aluminum alloy and stainless steel. Langmuir, 25(3):1620-1626.
Chou & Fasman (1974) Prediction of protein conformation. Biochemistry, 13:222-245.
Chou & Fasman (1978) Empirical predictions of protein conformation. Ann. Rev. Biochem., 47:251-276.
Chou & Fasman (1979) Prediction of beta-turns Biophys. J., 26:367-384.
Chowdhury et al. (2012) Combined factors influencing the aggregation and deposition of nano-tio2 in the presence of humic acid and bacteria. Environmental Science & Technology, 46(13):6968-6976.
Cook et al. (2017) Using the agricultural environment to select better surrogates for foodborne pathogens associated with fresh produce. International Journal of Food Microbiology, 262(Supplement C):80-88.
Corcoran et al. (2014) Commonly Used Disinfectants Fail To Eradicate Salmonella enterica Biofilms from Food Contact Surface Materials. Applied and Environmental Microbiology, 80(4):1507-1514.
Deutscher et al. (ed.) (1990) Guide to Protein Purification, Harcourt Brace Jovanovich, San Diego, Calif.
Devereux et al. (1984) A comprehensive set of sequence analysis programs for the VAX. Nucl. Acids Res. 12:387-395.
Di Bonaventura et al. (2008) Influence of temperature on biofilm formation by Listeria monocytogenes on various food-contact surfaces: Relationship with motility and cell surface hydrophobicity. Journal of Applied Microbiology, 104(6):1552-1561.
Duncan & Chang (2012) Chapter Two—implications of light energy on food quality and packaging selection. in Advances in Food and Nutrition Research, Henry (ed), Academic Press, New York, N.Y., pp. 25-73.
Ebbensgaard et al. (2018) The Role of Outer Membrane Proteins and Lipopolysaccharides for the Sensitivity of Escherichia coli to Antimicrobial Peptides. Front Microbiol 9:2153.
Eckersley & Berger (2018) An engineered polysaccharide lyase to combat harmful algal blooms. Biochemical Engineering Journal, 132:225-232.
Elasri & Miller (1999). Study of the response of a biofilm bacterial community to uv radiation. Applied and Environmental Microbiology, 65(5):2025-2031.
Eleftheriadou et al. (2017) Nanotechnology to the rescue: Using nano-enabled approaches in microbiological food safety and quality. Current Opinion in Biotechnology, 44:87-93.
Elimelech et al. (2013) Particle Deposition and Aggregation: Measurement, Modelling and Simulation. Butterworth-Heinemann Ltd. Oxford, England.
Foppen et al. (2008) Effect of humic acid on the attachment of Escherichia coli in columns of goethite-coated sand. Water Res 42:211-219.
Freireich et al., (1966) Quantitative comparison of toxicity of anticancer agents in mouse, rat, hamster, dog, monkey, and man. Cancer Chemother Rep. 50:219-244.
Genaro (ed.) (1985) Remington's Pharmaceutical Sciences, Mack Publishing Co., Easton, Pa.
Gerhardt et al. (eds.) (1994) Methods for General and Molecular Bacteriology, American Society for Microbiology, Washington, D.C., p. 574.
Gibson et al. (1999) Effectiveness of cleaning techniques used in the food industry in terms of the removal of bacterial biofilms. Journal of Applied Microbiology, 87(1):41-48.
Gross & Mienhofer (eds.) (1981) The Peptides, Vol. 3, Academic Press, New York, N.Y., pp. 3-88.
Havelaar et al. (2015) World health organization global estimates and regional comparisons of the burden of foodborne disease in 2010. PLOS Medicine 12(12):e1001923.
Haznedaroglu et al. (2009) Relative transport behavior of Escherichia coli O157:H7 and Salmonella enterica serovar pullorum in packed bed column systems: Influence of solution chemistry and cell concentration. Environmental Science & Technology, 43(6):1838-1844.
Herman et al. (2015) Outbreaks attributed to fresh leafy vegetables, united states, 1973-2012. Epidemiology and Infection, 143(14):3011-3021.
Hoff & Akin (1986) Microbial resistance to disinfectants: Mechanisms and significance. Environmental Health Perspectives, 69:7-13.
Hood & Zottola (1995) Biofilms in food processing. Food Control, 6(1):9-18.
Huang & Nitin (2017) Enhanced removal of Escherichia coli O157:H7 and Listeria innocua from fresh lettuce leaves using surfactants during simulated washing. Food Control, 79:207-217.
Izano et al. (2007) Detachment and Killing of Aggregatibacter actinomycetemcomitans Biofilms by Dispersin B and SDS. J Dent Res 86:618-622.
Jansen & Kohnen (1995) Prevention of biofilm formation by polymer modification. Journal of Industrial Microbiology, 15(4): 391-396.
Karlin & Altschul (1990) Methods for assessing the statistical significance of molecular sequence features by using general scoring scheme. Proc. Natl. Acad. Sci. USA 87:2264-2268.
Karlin & Altschul (1993) Applications and statistics for multiple high-scoring segments in molecular sequences. Proc. Natl. Acad. Sci. USA 90:5873-5877.
Kim & Harrison (2009) Surrogate selection for Escherichia coli O157:H7 based on cryotolerance and attachment to romaine lettuce. Journal of Food Protection, 72(7):1385-1391.
Kim et al. (2009) Spinocerebellar ataxia type 17 mutation as a causative and susceptibility gene in parkinsonism. Neurology, 72(16):1385-1389.
Kinsinger et al. (2017) Efficacy of post-harvest rinsing and bleach disinfection of E. coli O157:H7 on spinach leaf surfaces. Food Microbiology, 62:212-220.
Korber et al. (2009) 6—biofilm formation by food spoilage microorganisms in food processing environments. in Biofilms in the Food and Beverage Industries, Fratamico et al (eds.) Woodhead Publishing, Cambridge, England, pp. 169-199.
Kyte & Doolittle (1982) A simple method for displaying the hydropathic character of a protein. J. Mol. Biol., 157:105-13.
Macarisin et al. (2012) Role of curli and cellulose expression in adherence of Escherichia coli O157:H7 to spinach leaves. Foodborne Pathogens and Disease, 9(2):160-167.
MacDonald & Berger (2014) Insight into the role of substrate-binding residues in conferring substrate specificity for the multifunctional polysaccharide lyase smlt1473. Journal of Biological Chemistry, 289(26):18022-18032.
Madigan et al. (1997) Brock Biology of Microorganisms. Vol. 11. Prentice Hall, Upper Saddle River, N.J.
Maharjan et al. (2017) Effects of chlorine and hydrogen peroxide sanitation in low bacterial content water on biofilm formation model of poultry brooding house waterlines. Poultry Science, 96:2145-2150.
Martínez-Sánchez et al. (2006) Microbial, nutritional and sensory quality of rocket leaves as affected by different sanitizers. Postharvest Biology and Technology, 42(1):86-97.
Mayton et al. (2019a) Escherichia coli O157:H7 and Salmonella typhimurium adhesion to spinach leaf surfaces: Sensitivity to water chemistry and nutrient availability. Food Microbiol 78:134-142.
Mayton et al. (2019b) Influence of nano-CuO and —TiO₂ on deposition and detachment of Escherichia coli in two model systems. Environ Sci Nano 6:3268-3279.
McClaine & Ford (2002) Reversal of flagellar rotation is important in initial attachment of Escherichia coli to glass in a dynamic system with high- and low-ionic-strength buffers. Applied and Environmental Microbiology, 68(3):1280-1289.
Meireles et al. (2017) Comparative stability and efficacy of selected chlorine-based biocides against Escherichia coli in planktonic and biofilm states. Food Research International, 102:511-518.
Misra et al. (2015). “Bacterial Polysaccharides: An Overview”, in Ramawat & Mërillon (eds.) Polysaccharides: Bioactivity and Biotechnology. Springer International Publishing, Cham, Switzerland, pp. 81-108.
Ölmez & Kretzschmar (2009) Potential alternative disinfection methods for organic fresh-cut industry for minimizing water consumption and environmental impact. LWT—Food Science and Technology, 42(3):686-693.
Palmer et al. (2007) Bacterial cell attachment, the beginning of a biofilm. Journal of Industrial Microbiology & Biotechnology, 34(9):577-588.
Park & So (2000) Altered cell surface hydrophobicity of lipopolysaccharide-deficient mutant of Bradyrhizobium japonicum. Journal of Microbiological Methods, 41(3):219-226.
Patel et al. (2011) Effect of curli expression and hydrophobicity of Escherichia coli O157:H7 on attachment to fresh produce surfaces. Journal of Applied Microbiology, 110(3):737-745.
Pawar et al. (2005) Role of curli fimbriae in mediating the cells of enterohaemorrhagic Escherichia coli to attach to abiotic surfaces. Journal of Applied Microbiology, 99(2):418-425.
Pembrey et al. (1999) Cell surface analysis techniques: What do cell preparation protocols do to cell surface properties? Applied and Environmental Microbiology, 65(7):2877-2894.
Prigent-Combaret et al. (2001) Complex regulatory network controls initial adhesion and biofilm formation in Escherichia coli via regulation of thecsgD gene. Journal of Bacteriology, 183:7213-7223.
Rapicavoli et al. (2015) O antigen modulates insect vector acquisition of the bacterial plant pathogen xylella fastidiosa. Applied and Environmental Microbiology, 81(23):8145-8154.
Rosenberg et al. (1980) Adherence of bacteria to hydrocarbons: A simple method for measuring cell-surface hydrophobicity. FEMS Microbiology Letters, 9(1):29-33.
Ryu & Beuchat (2005) Biofilm formation by Escherichia coli O157:H7 on stainless steel: Effect of exopolysaccharide and curli production on its resistance to chlorine. Applied and Environmental Microbiology, 71(1):247-254.
Ryu et al. (2004) Attachment and biofilm formation by Escherichia coli O157:H7 on stainless steel as influenced by exopolysaccharide production, nutrient availability, and temperature. Journal of Food Protection, 67(10):2123-2131.
Saldaña et al. (2009) Synergistic role of curli and cellulose in cell adherence and biofilm formation of attaching and effacing Escherichia coli and identification of Fis as a negative regulator of curli. Environmental Microbiology, 11(4):992-1006.
Sharapov et al. (2016) Multistate outbreak of Escherichia coli O157:H7 infections associated with consumption of fresh spinach: United States, 2006. Journal of Food Protection, 79(12):2024-2030.
Slayton et al. (2013) Outbreak of shiga toxin-producing Escherichia coli (stec) O157:H7 associated with romaine lettuce consumption, 2011. Plos One, 8(2):e55300.
Solano et al. (2002) Genetic analysis of Salmonella enteritidis biofilm formation: Critical role of cellulose. Molecular Microbiology, 43(3):793-808.
Solomon et al. (2005) Biofilm Formation, Cellulose Production, and Curli Biosynthesis by Salmonella Originating from Produce, Animal, and Clinical Sources. J Food Prot 68:906-912.
Stewart et al. (1984) in Solid Phase Peptide Synthesis, 2nd Edition, Pierce Chemical Company, Rockford, Ill.
Suslow (2000) Postharvest handling for organic crops. Regents of the University of California, Division of Agriculture and Natural Resources, Davis, Calif.
U.S. Pat. No. 7,989,604.
Uhlich et al. (2008) Characterization of shiga toxin-producing Escherichia coli isolates associated with two multistate food-borne outbreaks that occurred in 2006. Applied and Environmental Microbiology, 74(4):1268-1272.
Van Houdt & Michiels (2010) Biofilm formation and the food industry, a focus on the bacterial outer surface. J Appl Microbiol 109:1117-1131.
Walker et al. (2004) Role of cell surface lipopolysaccharides in Escherichia coli K12 adhesion and transport. Langmuir, 20(18):7736-7746.
Walker et al. (2005) Influence of growth phase on adhesion kinetics of Escherichia coli D21g. Applied and Environmental Microbiology, 71(6):3093-3099.
Walsh et al. (2003) Activity and mechanisms of action of selected biocidal agents on Gram-positive and -negative bacteria. J Appl Microbiol 94:240-247.
Wang et al. (2013) Biofilm formation of meat-borne Salmonella enterica and inhibition by the cell-free supernatant from Pseudomonas aeruginosa. Food Control, 32(2):650-658.
Zhao et al. (2015) Bacterial cell surface properties: Role of loosely bound extracellular polymeric substances (LB-EPS). Colloids Surf B Biointerfaces 128:600-607.
Zogaj et al. (2001) The multicellular morphotypes of Salmonella typhimurium and Escherichia coli produce cellulose as the second component of the extracellular matrix. Molecular Microbiology, 39(6):1452-1463.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A polypeptide comprising having at least about 95% but less than 100% sequence identity to a polypeptide having an amino acid sequence as set forth in SEQ ID NO: 2 or SEQ ID NO: 4.

2. A polypeptide comprising an amino acid sequence that is a variant of the amino acid sequence of a wild-type Salmonella phage sequence set forth in SEQ ID NO: 2, wherein the variant sequence comprises at least one substitution at an amino acid position selected from the group consisting of D287, D291, D311, N313, D315, L307, and N284 of SEQ ID NO: 2 and wherein said polypeptide inhibits the growth of a microbe or microbial biofilm, and/or disrupts a microbial biofilm.

3. A nucleic acid molecule encoding the polypeptide of claim 1.

4. The nucleic acid molecule of claim 3, wherein the nucleic acid molecule is operably linked to a promoter.

5. The nucleic acid molecule of claim 4, wherein the nucleic acid molecule is a DNA segment, and the DNA segment and promoter are operably linked in a recombinant vector.

6. A recombinant host cell comprising the nucleic acid molecule of claim 3.

7. A recombinant vector, optionally an expression vector, comprising the nucleic acid molecule of claim 3.

8. A recombinant host cell comprising the recombinant vector of claim 7.

9. An antimicrobial composition comprising, consisting essentially of, or consisting of an effective amount of the polypeptide of claim 1 and a carrier.

10. An antimicrobial composition comprising, consisting essentially of, or consisting of an effective amount of the polypeptide of claim 2 and a carrier.

11. The antimicrobial composition of claim 10, further comprising one or more additional active agents, optionally wherein the one or more additional active agents are selected from the group comprising an additional antimicrobial agent, optionally an antibiotic and/or antifungal agent; a disinfectant, optionally a bleach; a pesticide, a fertilizer, an insecticide, an attractant, a sterilizing agent, an acaricide, a nematocide, an herbicide, and a growth regulator.

12. The antimicrobial composition of claim 7, wherein the polypeptide is present at a concentration in the range of from about 0.1 microgram per milliliter to about 100 milligrams per milliliter.

13. The antimicrobial composition of claim 9, wherein the antimicrobial composition has a pH in the range of from about 4.0 to about 9.0.

14. The antimicrobial composition of claim 9, wherein the antimicrobial composition is characterized by antimicrobial activity against E. coli, Salmonella, Pseudomonas, Listeria, Stenotrophomonas, and/or another pathogenic bacteria.

15. A method for inhibiting the growth of a microbe or a microbial biofilm on a surface, optionally a surface of an agricultural product or of a medical device, and/or for disrupting a microbial biofilm on the surface, the method comprising contacting the surface with an effective amount of an antimicrobial composition of claim 9.

16. A method for inhibiting the growth of microbe on and/or in a subject, the method comprising contacting the subject and/or administering to the subject the antimicrobial composition of claim 9 in an amount and via a route sufficient to inhibit the growth of the microbe on and/or in the subject.

17. The method of claim 16, wherein the microbe is a pathogenic bacterium, optionally a bacterium selected from the group consisting of E. coli, Salmonella, Pseudomonas, Listeria, and Stenotrophomonas.

18. The method of claim 16, further comprising contacting the subject and/or administering to the subject one or more additional active agents before, in conjunction with, and/or after contacting the subject and/or administering to the subject the antimicrobial composition of claim 9.

19. The method of claim 18, wherein the one or more active agents are selected from the group consisting of an additional antimicrobial agent, optionally an antibiotic and/or an antifungal agent; a disinfectant, optionally a bleach; a pesticide, a fertilizer, an insecticide, an attractant, a sterilizing agent, an acaricide, a nematocide, a herbicide, and a microbial growth regulator.
